(19) United States
(12) Patent Application Publication
(10) Pub. No.: US 2021/0393495 A1
(43) Pub. Date: Dec. 23, 2021

(54) FOAMABLE DENTAL CARE COMPOSITION, SYSTEM CONSISTING OF THE DENTAL CARE COMPOSITION AND A DISPENSER, AND USE OF THE DENTAL CARE COMPOSITION IN A METHOD FOR SIMULTANEOUSLY CLEANING SEVERAL, PREFERABLY ALL, TEETH

(71) Applicant: BLBR GmbH, Grünwald (DE)

(72) Inventor: Michael KEINER, Braunfels (DE)

(21) Appl. No.: 17/287,560

(22) PCT Filed: Nov. 22, 2019
(86) PCT No.: PCT/EP2019/082325

§ 371 Date: Apr. 22, 2021

(30) Foreign Application Priority Data
Nov. 23, 2018 (EP) 18208042.4

(51) Int. Cl. A61K 8/25; A61K 8/34; A61K 8/73; A61K 8/02; A61K 8/46; A61K 8/44; A61K 8/04; A61Q 11/00; A45D 40/00; B65D 83/62; A46B 9/04; A46B 13/02

(52) U.S. Cl. CPC A61K 8/25; A46B 2200/1066; A61K 8/73; A61K 8/0241; A61K 8/732; A61K 8/463; A61K 8/44; A61K 8/046; A61Q 11/00; A45D 40/00; B65D 83/62; A46B 9/045; A46B 13/023; A61K 2800/30; A61K 2800/28; A45D 2200/054; A61K 8/345

(57) ABSTRACT

The present disclosure relates to foamable dental care compositions and their use thereof for cleaning and caring of the teeth and mouth. With the preparation in foam or aerosol form, the composition can be applied evenly, quickly and efficiently on mouth inserts, cleaning prostheses and other devices used for the simultaneous cleaning of several or all teeth at the same time. The handling is considerably simplified.

### TECHNICAL FIELD

The present invention relates to a foamable mouth-, dental care and tooth cleaning composition, which is particularly suitable for use in tooth cleaning and tooth care by means of a tooth brushing device for the simultaneous cleaning of several, preferably all teeth of a user. Due to its low viscosity, the composition can be applied and distributed evenly, quickly and efficiently on the mouth insert of the toothbrush. The composition of the invention expands to short-term stable foam with good adhesiveness on the mouth insert, which can then be liquefied again. This makes handling much easier. Nevertheless, a very good and gentle cleaning of the teeth and gums is possible. The present invention further relates to the use of the foamable composition in a method tor simultaneous cleaning several, preferably all teeth by means of the tooth brush device. In addition, the present invention provides a system consisting of the dental care and tooth cleaning composition according to the invention and a dispenser. With the help of this system, the composition can be applied to the mouth insert in the desired amount and expand therein further into a foam.

### STATE OF THE ART

Generally, oral, and dental care products are available in paste or cream form. They have a viscosity of 60,000 to 80,000 mPas (millipascal seconds; measured at 20° C.) and are released from tubes or stand containers by manually squeezing the container onto the brush head of a toothbrush. The usual application amount is 1 to 2 ml per use. In rare cases, oral and dental care products in gel form with a viscosity of 10,000 to 70,000 mPas (at 20° C.) are available. Again, an amount of 1 to 2 ml is applied to the brush head of a toothbrush by pressing on the dispenser container. A toothpaste with relatively low viscosity—in other words, a liquid—or fluid toothpaste—is disclosed in EP 2 813 214 A1.

This composition was formulated to allow a stable incorporation of amine fluorides, but still to be sufficiently viscous to be applied to brush heads of conventional- or electric toothbrushes. A tooth cleaning agent foaming after discharge from a storage container is disclosed in EP 1 257 250 B1. This composition is also formulated for use with conventional toothbrushes and therefore has a viscosity of more than 30,000 mPas.

However, the present invention is not directed to the application of the oral and dental care and cleaning composition with a conventional hand toothbrush or a common electric toothbrush. Rather, there are more recent developments in the field of tooth cleaning, in which it is no longer worked with usual brush heads, but several or all teeth of the user can be cleaned simultaneously by means of a mouth insert. These mouth inserts (see e.g., FIG. 1) are at least coarse, preferably individual, adapted to the teeth and jaw shape of a user and can clean several or even all teeth simultaneously, stimulated by a motor to vibrations. Corresponding tooth cleaning systems are disclosed in DE 10 2015 109891 A1, WO 2009/137671 A1 or WO 2015/003681 A1.

For use in such mouth inserts, which serve for the simultaneous cleaning of several or all teeth, the dosage forms of tooth cleaning and care products known in the prior art, such as toothpastes, dental creams or tooth gels, but also the known foams, are unsuitable. The highly viscous products would have to be distributed with a brush or a spatula on the cleaning surfaces of the mouth inserts, whereby the high viscosity of conventional toothpastes, dental creams or gels makes an even distribution almost impossible. At the same time, the amount of cream, paste or gel to be used would increase to about 20 to 25 ml per cleaning process, resulting in excessive waste of resources and an overdose. An overdose can lead, among other things, to too much dental abrasion or to damage of the dentine along the gum line.

A simple dilution of conventional dental care products may solve the problem of overdose, but it does not solve the problem of providing a product that takes into account the other special requirements when using one of the mouth inserts described above. A diluted dental cream, for example, does not adhere to the mouth inserts stable enough, especially in case of an overhead position, and would thus run out of the inserts even before they can be inserted into the mouth.

Even the foams known from the prior art do not meet the requirement profile for such a tooth cleaning composition. One reason for this is that the known foams are too stable and therefore cannot decay sufficiently quickly or be crushed by the user's teeth. The high foam stability is detrimental for several reasons. On the one hand, such a stable foam swells out of these for the most part when inserting the mouth inserts, which is undesirable and means a loss of active substances and thus a reduced cleaning effect. On the other hand, the foam, remaining on the mouth inserts, i.e. not displaced by the teeth, and still contains many air bubbles, which decrease or make the transmission of the vibrations of the motor difficult, which ultimately negatively affects the cleaning performance.

There is therefore a need for novel tooth cleaning compositions, which are suitable for use with tooth brushing devices for simultaneous cleaning of all teeth of a user, thus taking into account the special requirements when using such tooth brushing devices. The compositions must be applied simply and evenly on appropriate mouth inserts or mouth insert elements and adhere there sufficiently and stable so that they do not emerge from the mouth inserts before they have been inserted into the mouth. At the same time, however, the foam must again be so unstable that it disintegrates again after insertion of the mouth inserts and/or can be crushed through the teeth. In addition, the formulations must ensure a stable dispersion of the abrasives or cleaning substances in the composition. This is the only way to ensure that they are evenly distributed in the mouth inserts and that all teeth can be cleaned gently and efficiently. Also, the compositions must meet the other requirements (plaque reduction, caries prophylaxis, etc.) which are placed on conventional toothpastes.

The conventional methods for cleaning the teeth with hand or electric tooth-brushing are not only time-consuming, because, due to the dimension of the brushing-head, the teeth have to be cleaned one after the other, they also carry the risk that either individual teeth or teeth areas are cleaned less well, because they are forgotten by the user during cleaning or not cleaned long enough, or that individual teeth or teeth are cleaned too long and too intensively and get thus damaged. Procedures for the simultaneous cleaning of all teeth thus improve the success of the cleaning and care measure and increase user compliance, especially by saving time.

The above problems were solved by the present invention.

### DESCRIPTION OF THE INVENTION

The present invention relates according to a first aspect of a dental care and tooth cleaning composition for use in a method for simultaneous cleaning of several, preferably all teeth of the upper and/or lower jaw, by means of a suitable tooth brushing device, wherein the dental care and tooth cleaning composition is presented in foamed form on a mouth insert or several mouth inserts the tooth brushing device, and wherein the tooth care and tooth cleaning composition comprises as essential components:
10 to 40 wt.-%, preferably 15 to 30 wt.-%, glycerin.
10 to 23.5 wt.-% at least one precipitated silicate, preferably 10 to 25 wt.-% of a mixture of 2 to 4 precipitated silicates,
0.05 to 0.5 wt.-% at least one viscosity amplifier,
at least one surfactant, preferably with a proportion of 0.2 to 4.0 wt.-% of one or more surfactant(s), and
at least 10 wt.-% water.

The mouth insert or mouth inserts of the tooth brushing device is/are at least coarse, preferably individual, adapted to the denture and jaw shape of a user and has/have one or more recess for receiving the teeth. According to a possible embodiment, the mouth insert is formed in one piece, that is, the mouth insert is designed so that it is suitable for the simultaneous reception of the teeth of the upper and the teeth of the lower jaw (see FIG. 1). The recesses for receiving the totality of the teeth are thus located on facing away sides of the mouth insert or open in opposite directions. Alternatively, the tooth brushing device can be several, preferably two mouth inserts, wherein the first mouth insert for receiving the teeth of the upper jaw and the second mouth insert for receiving the teeth of the lower jaw is designed. Even further alternatively, the tooth brushing device can be designed in such a way that it has only one mouth insert, which is designed only to receive the teeth of a jaw, but is designed so flexible that it can adapt to the teeth of the upper jaw as well as to the teeth of the lower jaw. In such a case, first the tooth row of one jaw and then the tooth row of the other jaw would be cleaned. Even further alternatively, the mouth insert or the mouth inserts can be designed so that they are each designed for the reception of tooth areas of the upper jaw and/or the lower jaw, wherein the tooth areas are formed by the adjacent teeth in the jaw, for example the molars or the incisors. For simultaneous tooth cleaning, several such oral inserts are used at the same time, which must be selected in such a way that all teeth of the upper and/or lower jaw are received. Alternatively, the cleaning of all teeth could also be done in a time-shifted manner. Preferred embodiments of the oral inserts are described in detail below, also with reference to FIGS. 1 to 5. All these oral inserts are particularly suitable for the administration of the dental care and tooth cleaning composition according to the first aspect of the invention.

Preferred embodiments of the inventive use are the use in a method for the prevention and/or treatment of dental and gum diseases, in particular of caries, periodontitis and plaque, by the simultaneous cleaning of several, preferably all teeth of the upper and/or lower jaw of a user.

The term “dental care- and tooth cleaning composition” is used here and is used in the following below synonymously with the term “formulation” or “composition”.

In the sense of the present invention, the simultaneous cleaning of several teeth means that the mouth insert or the mouth inserts are formed/that preferably at least 3, even more preferably at least 5, even more preferably at least 7 teeth can be cleaned. Particularly preferred is the mouth insert or the mouth inserts are formed so that at the same time all teeth of the upper jaw and/or the lower jaw can be cleaned. At the same time, the mouth insert or mouth inserts at a time T1 are in contact with all teeth to be cleaned and clean them. In contrast, the brush head of a conventional manual or electric tooth brush comes into contact with the teeth to be cleaned one after the other. This not only increases the time required for cleaning, but also carries the risk that not all teeth are cleaned with the same care and intensity.

The essential components of the composition used according to the invention in the specified proportion ranges allow the provision of a low viscous composition.

An essential component of the composition is glycerin. Although it was generally known in the prior art that glycerin can be used as a possible humectant in dental cleaning and dental care products. At the same time, however, the relevant state of the art has provided the skilled person with a wide range of other possible humectants. Humectants are usually added to dental care and teeth cleaning products to prevent the composition from drying out. It has now been surprisingly found that glycerin, when used in a high proportion, can be used as a matrix former for the remaining substances and as a stabilizer for the composition, in particular as a short-term stabilisator for a foam obtained from the composition. The beneficial multiple suitability of glycerin was surprising.

Without wanting to be bound to this theory, it appears that the glycerin lowers the surface tension of the water also contained in the composition with a relatively high proportion. This has the consequence, on the one hand, that the stability of a foam obtained from the composition is influenced so that for the time window of application of the composition on the mouth insert or the mouth inserts a high foam stability and adhesion of the foam can be achieved. In practice, this time window is usually in a range of a few seconds. In addition, this advantageous combination of the said components also causes that on the other hand, after insertion of the mouth inserts into the mouth, the foam becomes so unstable again during application, i.e. during dental care and tooth cleaning, that it quickly disintegrates sufficiently quickly again more or less into a liquid form. In this case, the mechanical influence of the teeth can also be conducive, which additionally crush the foam to the recesses of the mouth inserts when inserting the same.

Furthermore, it was surprisingly found that by the use of one or more precipitated silicates the composition as such, but also the foam obtained from the composition can be advantageously stabilized. This or the precipitated silicates therefore function not only as cleaning substances, but also as a stabilizer for dispersion and as a foam stabilizer. The thus obtained foam is not only temporarily stable and destructible again, but it is also characterized by a good adhesion also in the overhead position of a mouth insert.

The cleaning substance of a dental care and tooth cleaning composition, which can also be called abrasive or polishing agent, is basically an abrasive and allows an effective removal of plaques during brushing teeth. Water-insoluble inorganic substances are usually used as cleaning substances, which mechanically remove the plaque without damaging the enamel or dentine. As with the humectants, the relevant state of the art has given the skilled person a very wide range of possible cleaning substances. It was surprising that by the selection of one or more precipitated silicates as cleaning substances, in particular with a simultaneously high proportion of glycerin of at least 10 wt.-%, preferably at least 15 wt.-%, and a water content of at least 10 wt.-% the formulations can be stabilized as such and in addition, short-term stable, well-adhesive foams can be provided.

The application of a low viscous and foamable composition allows the easy and precise application of the composition to a mouth insert, where it can expand further into a foam. The expansion, i.e. the foam formation, can be accomplished by the use of one or more propellants. For this purpose, the composition is provided in a suitable container, preferably an aerosol can or a bag-on-valve can. Details are described below.

In a preferred explanation of the first aspect of the present invention, the composition is formulated in such a way that the liquid pre-formulation, i.e. the composition without propellant, has a viscosity in the range of 200-30,000 m Pas. Even more preferably, the viscosity of the composition without propellant gas is in a range of 400-10,000 m Pa s and most preferably at less than 10,000 m Pa s. For example, a viscosity in the range of 500-9,000 m Pa s is even more preferred. Even more preferred is the viscosity in a range of 1,000-8,000 m Pa s. The selected viscosity range ensures that the composition can be applied evenly and easily to the mouth insert or the mouth inserts of the tooth brushing device and also adheres to this/these for enough long until foaming.

The proportion of one or more surfactants in the composition is preferably 0.35 to 3.0 wt-%, even more preferably 0.6 to 2.5 wt.-%. Particularly advantageous surfactants as well as preferred precipitated silicates, viscosity amplifiers and other components of the composition are described below in connection with the foamable dental care- and tooth cleaning composition as such. This disclosure is also to apply expressly to the first aspect of the invention described above.

The present invention relates according to a second aspect to a foamable dental care and tooth cleaning composition as such. The composition according to the second aspect of the present invention is advantageously adapted to the special requirements when used in a mouth insert for simultaneous cleaning of several, preferably all teeth of the upper and/or lower jaw, according to the first aspect of the present invention.

The dental care and cleaning composition of the invention comprises as essential components:
15 to 30 wt.-%., preferably at least 20 wt.-%, even more preferably at least 22 wt.-%, glycerin,
optionally at least one other humectant, in addition to glycerin, wherein the at least one other humectant is preferably sorbitol or hydrogenated starch hydrolysate or comprises,
10 to 23.5 wt.-% at least one precipitated silicate,
0.2 to 4.0 wt.-% at least one surfactant,
0.05 to 0.5 wt.-% at least one viscosity amplifier, and
at least 10 wt.-% water.

The formulation “at least one precipitated silicate”, means in the sense of the present invention that the composition of the invention contains a silicate component—in the specified proportion range—and this silicate component is either a precipitated silicate/silicate-type or the silicate component is a mixture of several precipitated silicates/silicate-types. The same applies to the formulations “at least one other humectant,” at least one surfactant”, “at least one viscosity amplifier”, etc.

The composition according to the composition of the invention according to the second aspect of the present invention as well as the composition for use according to the first aspect of the present invention contains as an essential component glycerin and water with a proportion of at least 10 wt.-%. As already explained in connection with the first aspect of the present invention, it was surprisingly found that in a composition with at least 10 wt.-% glycerin, the glycerin not only acts as a humectant, but in combination with the high water content also as a matrix for the remaining substances and as a stabilizer for the composition, in particular as a short-term stabilizer for a foam available from the composition.

The component glycerin is therefore included in the composition of the invention with a proportion of 15 to 30 wt.-%, based on the total composition. Preferably, the proportion of glycerin in the total composition is at least 20 wt.-%, even more preferably at least 22 wt.-%. The upper limit of the glycerin-proportion is preferably 28 wt.-%. According to the invention particularly preferred range are, for example, 20 to 30 wt.-% or 22 to 28 wt.-% glycerin, based on the total composition.

In addition to the mentioned amounts or proportions of glycerin, the composition of the invention may contain further humectants. The or the further humectants serves or serve, like the glycerin, on the one hand to prevent a drying out of the composition. However, the other humectant agent can also act as a consistency provider and as a cold stabilizer.

Pharmaceutically and/or cosmetically acceptable humectants are generally known to the skilled person. In a preferred embodiment according to the invention, the composition comprises as further humectant sorbitol, hydrogenated starch hydrolysate, xylitol or any mixtures of these preferred moisturizers. Further, known in the prior art humectants may additionally be included in these preferred humectants.

The proportion of the further humectant is in wt.-%, based on the total composition, is preferably to be chosen so that the proportion of glycerin plus the proportion or the proportions of further humectant agents is in the total range of 30 to 70 wt.-%, Preferably, the total proportion is 31.25 to 65 wt.-%, even more preferably 40 to 62 wt.-%, most preferably 40 to 60 wt.-%, each based on the total composition.

The at least one precipitated silicate contained in the composition of the invention serves, as already mentioned above, among other things as a cleaning substance.

In the compositions of the invention, the cleaning substance may contain a substance or a mixture of different substances.

The compositions of the invention contain as cleaning substances at least a compound from the group of silicates, which in the relevant prior art are referred to as precipitated silicates (“hydrated silica”). Suitable precipitated silicates are commercially available and known to the skilled person.

The precipitated silicates preferably have an average particle size in the range of 1 to 50 microns, even more preferably from 6 to 14 microns.

Furthermore, the precipitated silicates preferably have a Einlehner abrasion in the range of less than 2.0 to 30 mg loss/100 000 revolutions. In trade precipitated silicates are available, which differ in their average particle size and abrasiveness. According to the invention particularly suitable precipitated silicates are available, for example, under the designation ZEODENT® 103, ZEODENT® 115, ZEODENT® 165 and ZEODENT® 167. While ZEODENT® 103 has a relatively strong abrasive effect and has the smallest mean particle size compared to the other ZEODENT forms mentioned, ZEODENT® 15 has a significantly lower abrasion value. Even lower is the abrasion value of ZEODENT® 165 and ZEODENT® 167, but these have a higher mean particle size. To the extent of the present invention, also any mixtures of these exemplary precipitated silicates are included. A goal of the use of precipitated silicates or any mixture of such is to provide a medium to high abrasion factor of dental care and tooth cleaning composition.

The abrasion factor of the composition is preferably given as the RDA value of the composition. “RDA” stands for radioactive dentin abrasion. The RDA value of the compositions of the invention is preferably in a range of 30 to 160. At an RDA value of less than 30, the cleaning effects of the composition are lower, while at an RDA value of more than 160 there is a risk that the composition is so abrasive that it can damage the tooth leg along the gum line. Particularly preferred is the RDA value of the composition of the invention in a range of 50 to 130. In this area, the gentle cleaning effect achieved is particularly advantageous. The abrasion factor and in particular the RDA value of a tooth cleaning composition depends both on the abrasiveness of the cleaning substances used and on their concentration in the composition. Therefore, the precipitated silicates are added in a sufficient quantity and, if necessary, in an appropriate mixing ratio to adjust the desired abrasion factor.

The precipitated silicates in the composition of the invention do not only act as cleaning substances, but they also have, at least in part, thickening properties. In a preferred embodiment of the present invention, the amount of thickening precipitated silicates is at least 1 wt.-%, preferably at least 1.5 wt.-%, even more preferably at least 3 wt.-%, based on the overall composition. The upper limit on such thickening precipitated silicates is 10 wt.-%, preferably 7 wt.-%, even more preferably 3.5 wt.-% or 3 wt.-%.

The at least one precipitated silicate is comprised with a proportion of 10 to 23.5 wt.-%, based on the total composition, in the composition of the invention. Preferably, the proportion of the at least one precipitated silicate is 12 to 23 wt.-%, even more preferably 14 to 22 wt.-%, further preferably 16 to 21 wt.-%, each based on the total composition. The lower limit of the proportion range is most preferably 17 or 18 wt.-%.

Furthermore, the cleaning substance of the composition of the invention may comprise at least one of the following substances. Silicates, such as various aluminum silicates and zirconium silicates, but also sodium aluminum silicates, such as synthetic zeolites. Silica Dimethyl Silylate or alumina oxide are also conceivable. These substances may be present individually or in any mixtures thereof. Further, known in the prior art cleaning substance substances may additionally be included in this preferred cleaning substance.

In an alternative embodiment of the composition according to the invention, it contains no aluminum silicates, zirconium silicates, sodium aluminum silicates, such as synthetic zeolites, silica dimethyl silylate and/or aluminum oxide. Apart from unavoidable impurities and in the range of the usual measuring accuracy, the proportion of these compounds acting as cleaning substances is 0 wt.-%.

The composition of the invention further has at least one surfactant. The term “surfactant” is used here and herein after synonymously with the term “surface-active substance”. Pharmaceutically and/or cosmetically acceptable surface-active substance are generally known to the skilled person. For example, anionic, cationic, non-ionic or amphoteric surface-active substance can be used for this purpose. Exemplary examples are 1-hexadecanol, 1-octadecanol, polyacrylic acid, polyethylene glycol, cocamidopropyl betaines, sodium lauryl sulfate, sodium laurylsarcosides, trisodium-phosphate, sodium-methylcococyltaurate, fatty alcoholpolyglycosides, for example C₆-C₂₀-alkylgglycosides, individually mentioned or in any mixtures thereof.

In a preferred embodiment according to the invention, the composition as a surfactant comprises sodium lauryl sulfate and/or sodium lauryl sarcosinate. In another preferred embodiment, the composition includes cocamido-propyl betaines as a surfactant. Another preferred embodiment contains a mixture of sodium lauryl sulfate and cocamidopropyl betaine as surfactants. The weight ratio of sodium lauryl sulfate and cocamidopropyl betaine to each other lies preferably in the range of 4:1 (i.e. 4 parts sodium lauryl sulfate and 1 part cocamidopropyl betaines) to 1:4, even more before-increased in the range of 3:1 to 1:3, and most preferably in the range of 2.5:1 to 1:2.5.

According to a further preferred embodiment, the composition of the invention as a non-ionic surface-active substance comprises a C₆-C₂-alkylglykoside, preferably a C₈-C₁₆-alkylglykoside. Such alkyl glycosides are available in stores under the brand name Plantacare, for example. Even more preferably, the C₆-C₂₀-alkylglykoside, preferably the C₈-C₁₆-alkylglyoside, in combination with sodium lauryl sulfate and/or sodium lauryl sarcosinate. In another preferred embodiment is the C₆-C₂₀-alkylglykoside, preferably the C₈-C₁₆-alkylglykoside, in combination with cocamidopropyl betaines. In a further preferred embodiment, the composition of the invention contains a mixture of sodium lauryl sulfate, cocamido-propyl betaines and a C₆-C₂₀-alkylglykoside, preferably a C₈-C₁₆-alkyl-glycoside, as surfactants. The weight ratio of sodiumlauryl sulfate, cocamidopropyl betaines and C₆-C₂₀-alkylglyoside, preferably the C₈-C₁₆-alkylglykoside, to each other lies preferably in the range of 0.5-1.5 parts sodium lauryl sulfate, 0.1-1.0 parts cocamidopropyl betaines and 0.4-1.8 parts of the C₆-C₂₀-alkylglykoside, preferably of the C₈-C₁₆-alkylglykoside. Even more preferably, the weight ratio is in the range of 0.8-1.2 parts sodium lauryl sulfate, 0.2-0.8 parts cocamidopropyl betaines and 0.6-1.4 parts of the C₆-C₂₀-alkylglykoside, preferably of the C₈-C₁₆-alkylgglycoside.

The surfactants in the composition of the invention have a function in the formation of foam as well as in the regulation of the viscosity of the composition.

The proportion of at least one surfactant in the composition of the invention is 0.2 to 4.0 wt.-%, preferably 0.35 to 3.0 wt.-%, even more preferred 0.6 to 2.5 wt.-%, based on the overall composition. Alternatively, the proportion of at least one surfactant in the composition of the invention may be 0.5 to 3.0 wt.-%, based on the total composition.

The compositions of the invention further comprise at least one viscosity amplifier. For dental cleaning and care products basically suitable viscosity amplifiers include exemplary cellulose, cellulose derivatives, plants-gums, such as Xanthan Gum, pectins, carbomers, polysaccharides from algae, for example carrageenan, polyvinyl alcohol, polyvinylpyrrolidone, starch and starch ether. Cellulose derivate are, for example, hydroxyalkyl cellulose, such as hydroxyethyl cellulose, carboxymethylcellulose, hydroxyethyl cellulose, methyl-hydroxypropyl cellulose and their salts, for example sodium salts. According to one embodiment, the viscosity amplifier is selected from the group consisting of carboxymethyl cellulose, hydroxyethyl cellulose, Xanthan Gum, acrylates/C10-30-alkyl acrylate cross polymer and any mixtures thereof. The proportion of at least one viscosity amplifier in the composition according to the invention is 0.05 to 0.5 wt.-%, based on the total composition. Preferably, the proportion of at least one viscosity amplifier is 0.1 to 0.4 wt.-%, even more preferably 0.1 to 0.3 wt.-%, each based on the total composition.

The composition of the invention still comprises at least one solvent. The solvent is preferably water. Alternatively, the solvent may comprise water in a solvent mixture. The water is suitable for cosmetic or pharmaceutical formulations. Other pharmaceutically and/or cosmetically acceptable solvents are known to the skilled person.

The water is comprised with a proportion of at least 10 wt.-% in the composition and forms the rest to 100 wt.-% (ad, 100 wt.-%), based on the total composition. That is, the wt.-% portions of the essential and optional components, including further optional solvents other than water, the composition are always chosen in such a way that in total 100 wt.-% result.

The indication “the remainder to 100 wt.-%” or “ad. 100 wt.-%” is not to be understood as that in the composition of the invention optionally no further components, for example active substances and/or auxiliary or additives, as optional component(s) can no longer be included. The presence of further optional—also not explicitly named—components is expressly included according to the invention. If such components are to be excluded, the phrase “consisting of” is used here and below. The “rest” always represents the proportion of water that is necessary after sum-up of all essential and, if available, optional components, in order to then yield 100 wt.-%.

The proportion of water in the composition of the invention is preferably 10 to 40 wt.-%. Even more preferably, the limit of the water content is 12 wt.-% and/or the upper limit at 30 wt.-%, more preferably at 25 wt.-% and further preferably at 23 wt.-%. The water content can therefore, for example, according to the invention lie in the range of 12 to 23 wt.-%.

The composition according to the invention is suitable for cleaning and care of the teeth, the dentine and the gums and in particular for the prevention and/or treatment of dental and gum diseases, in particular of caries, periodontitis and plaque. In particular, the composition of the invention is suitable for application in tooth cleaning and care by means of a tooth brushing device for the simultaneous cleaning of several, preferably all teeth of a user. In this case, the composition according to the invention is presented by applying the composition to a mouth insert or several mouth inserts of the tooth-cleaning device.

According to a preferred embodiment, the composition of the invention comprises as a further humectant starch hydrolysate or the further humectant is hydrogenated starch hydrolysate. Hydrogenated starch hydrolysates (Hydrogenated Starch Hydrolysates) are mixtures of polyvalent alcohols such as sorbitol, maltitol and higher-order sugar alcohols. An inventive-according to particularly preferred hydrogenated starch hydrolysate contains between 65 and 75 wt-% sorbitol. Corresponding products are commercially available.

The proportion of hydrogenated starch hydrolysate in the total composition shall in any case be chosen in such a way that the amount of glycerin and hydrogenated starch-hydrolysate does not exceed the proportion of 70 wt.-%. Even more preferably, the amount of glycerin and hydrogenated starch hydrolysate in the range of 40 to 70 wt.-%, even further preferred in the range of 40 to 60 wt.-%. It is also provided according to the invention that the humectant component of the invention-corresponding composition may consist of glycerin and hydrogenated starch hydrolysate.

Irrespective of whether the humectant component consists of glycerin and hydrogenated starch hydrolysate or comprises this, the proportion of hydrogenated starch hydrolysate is preferably in the range of 25 to 40 wt.-%, even more preferably in the range of 28 to 38 wt.-%. Alternatively, the weight-ratio of hydrogenated starch hydrolysate, in particular hydrogenated starch hydrolysate with a sorbitol content in the range of 65 to 75 wt.-%, to glycerin preferably in a range of 0.5:1 to 5:1, may be even more preferably in a range of 0.8:1 to 3:1.

According to another preferred embodiment, the composition of the invention comprises as a further humectant agent sorbitol or the further humectant is sorbitol. It is therefore also provided according to the invention that the humectant component of the composition of the invention consists of glycerin and sorbitol.

The proportion of sorbitol according to an embodiment is preferably in the range of 16.25 to 30 wt.-%, even more preferably in the range of 20 to 28 wt.-%, even more preferably in the range of 20 to 26 wt.-%. Alternatively, the proportion of sorbitol in the composition of the invention can be 18.75 to 26 wt.-%. Irrespective of whether the humectant agent component consists of glycerin and sorbitol or comprises these two components, the ratio of sorbitol to glycerin is preferably in a range of 0.5:1 to 5:1, even more preferably in a range of 0.8:1 to 3:1.

In a preferred embodiment, the composition of the invention is thus free of xylitol. In the sense of the present invention, the formulation “free of xylitol” means that the composition xylitol has at most a proportion of less than 0.01 wt.-%, based on the total composition without propellant. The proportion of xylitol is thus in the range of 0 wt.-% to less than 0.01 wt.-%, based on the total composition without propellants. Preferably, the maximum permissible upper limit is 0.008 wt.-%, even more preferably 0.006 wt.-% and even further preferably 0.004 wt.-%, each based on the total composition without propellant. The proportion of xylitol in the composition 0 wt.-% is highly preferred. Any unavoidable impurities should be disregarded. The usual measurement accuracy also applies.

By selecting the above-mentioned components in the said quantities, a composition with low viscosity can be provided. The low viscosity of the composition allows a uniform and economical distribution of the composition on all cleaning surfaces of the mouth insert or mouth inserts. There is no overdose of cleaning substances, which could lead to mechanical damage to the enamel and an undesirable abrasion of the teeth, nor foams the composition too much or forms a foam that is too stable, no longer destructible.

By combining the above-mentioned essential components, thus a composition is made possible, which is particularly suitable for uniform, economical and simple application to the mouth insert or the mouth inserts of the toothbrush-device. By combining the above-mentioned essential components, the desired viscosity can be set stable and, in addition, a short-term stable foam formation can be achieved during the application.

According to a preferred embodiment of the composition according to the invention, it has a dynamic viscosity of 200-30,000 m Pa s without propellant gas. Preferably, the viscosity of the composition without propellant is in a range of 400-10,000 m Pa s and even more preferably at less than 10,000 m Pa s. For example, a viscosity in the range of 500-9,000 m Pa s at 20° C., is even more preferred. Even more preferred is the viscosity in a range of 1,000-8,000 m Pa s at 20° C. The selected viscosity range ensures that the composition according to the invention can be applied evenly and easily to the mouth insert or the mouth inserts of the tooth brush device and also adheres sufficiently long to it/them.

According to a preferred embodiment, the composition of the invention contains a mixture of at least 2, preferably 2 to 4 of the above in detail described precipitated silicates. Even more preferably, the mixture contains three precipitated silicates. The weight ratio of the three precipitated silicates is preferably in the range of 5-15 parts of a first silicate, 2-8 parts of a second silicate and 0.5-5 parts of a third silicate.

For example, such precipitated silicates, which are also characterized by their thickening properties, can be combined with those precipitated silicates, which are particularly suitable as cleaning substances.

As explained above, the precipitated silicates differ in their mean particle size and abrasiveness (hardness). In the context of the present-present disclosure, the precipitated silicates are therefore divided into three categories 1 to 3.

Here, precipitated silicates of category 1 are those that have an average particle size in a range of 7 to 8.5 microns and a Einlehner abrasion or a brass-Einlehner (BE) abrasion value in the range of more than 5.0 to 25 mg loss/100 000 revolutions, preferably in the range of 10-20 mg loss/100 000 revolutions. An example of a type 1 silicate is ZEODENT® 103. Precipitated silicates of category 2 are defined as having an average particle size in a range of more than 8.5 to 10 microns and a Einlehner abrasion in the range of more than 2.5 to 5.0 mg loss/100 000 revolutions, preferably in the range of 2.7 to 4.0 mg loss/100 000 revolutions. An example of a type 2 of the case is ZEODENT® 115. Precipitated silicates of category 3 are defined as those that show an average particle size in a range of more than 10 to 14 microns and a Einlehner abrasion in the range of less than 2.5 loss/100 000 revolutions, preferably in the range of 0.1 to 2.3 mg loss/100 000 revolutions. Examples of category 3 silicates are ZEODENT® 165 and ZEODENT® 167.

According to the invention, therefore, mixtures of at least one precipitated silicate of category 1, for example ZEODENT® 103, at least one precipitated silicate of category 2, for example ZEODENT® 115, and at least one precipitated silicate of category 3, for example ZEODENT® 165 and/or ZEODENT® 167.

A goal of the use of any mixture of precipitated silicates and in particular-especially of precipitated silicates from categories 1 to 3, is to provide a medium to high abrasion factor of dental care and tooth cleaning composition and a suitable thickening of the composition. The RDA value of the compositions of the invention is preferably in a range of 30 to 160, even more preferably in a range of 50 to 130.

Since the abrasion factor and in particular the RDA value of a tooth cleaning composition depends both on the abrasiveness of the used precipitated silicate and on their proportion in the composition, the precipitated silicates are preferably with a weight ratio of 5-15 parts at least one precipitated silicate of category 1, For example, ZEODENT® 103, 2-8 parts at least one precipitated silicate of category 2, for example ZEODENT® 115, and 0.5-5 parts at least one precipitated silicate of category 3, for example ZEODENT® 167, contained in the mixture of the precipitated silicates. Even more preferably with a weight ratio of 7-13 parts at least one precipitated silicate of category 1, for example ZEODEN® 103,3-7 parts at least one precipitated silicate of category 2, for example ZEODENT® 115, and at least one precipitated silicate of category 3, for example 1,5-3,5 parts ZEODENT® 167. Likewise, it is preferred according to the invention that the at least one precipitated silicate of category 1, for example ZEODENT® 103, with a proportion of 6 to 15 wt.-%. preferably with a proportion of 7 to 13 wt.-%, that at least one precipitated silicate of category 2, for example, ZEODENT® 115, with a proportion of 3 to 7 wt.-%, and that at least one precipitated silicate of category 3, for example ZEODENT® 167, with a proportion of 1 to 3.5 wt.-%. preferably with a proportion of 1.5 to 3 wt.-%, is/are contained composition according to the invention.

In addition to the above-mentioned precipitated silicates, the composition of the invention may also include one or more pyrogenic silicates. Pyrogenic silicates are not produced wet chemically, but by a thermal process, preferably by flame hydrolysis and can be used without further grinding or spray drying.

The specific surface according to BET of the pyrogenic silicates is preferably in the range of 10 to 1000 m/g, even more preferably in the range of 20 to 800 m²/g and highly preferably in the range of 40 to 600 m²/g. The average primary particle size, i.e. the size of the smallest components, which are more or less strongly aggregated and agglomerated in the pyrogenic silicate, is preferably in the range of 2 to 70 nm, even more preferably in the range of 5 to 50 nm.

The pyrogenic silicates obtained directly after the thermal process are hydrophilic due to the freely accessible silanol groups (Si—OH) on the particle surface and can thus be wetted with water. In one embodiment of the present invention is the/are the pyrogenic (s) silicate (s) in addition to one or more precipitated silicates contained in the composition of the invention hydrophilic. Hydrophilic pyrogenic silicates are commercially available and can be obtained, for example, under the name HDK® N20.

According to the invention, however, it is also provided in another embodiment that the pyrogenic (s) silicate (s) contained in the composition of the invention in addition to the one or the several precipitated silicates are surface-treated and thus has/have hydrophobic properties. The surface treatment is preferably carried out by a chemical post-treatment-step. The hydrophobic behavior thus produced is caused by the reaction of hydrophilic silanol groups with organic residues, preferably with silanes, for example, dichlorimimethylsilane, alkoxy silane, silazanes, siloxanes, C₁-C₅ carbon groups, for example CH₃ groups. Hydrophobic pyrogenic silicates are commercially available and can be purchased under the name Aerosil R 812 S, for example.

According to a further embodiment of the present invention, the composition may also contain a mixture of at least one hydrophobic and at least one hydrophilic pyrogenic silicate.

By the use of at least one pyrogenic silicate, the composition in its thixotropic behavior can be advantageously adjusted. Thixotropy refers to a time-dependent change in the flow properties of fluids, in which the viscosity decreases or increases as a result of persistent external influences and only returns to the initial viscosity after the end of the application.

This makes the composition with the duration of its deformation thinner. Thus, it is ensured that with low mechanical stress or load, for example during storage, the viscosity of the composition is high enough to prevent settlement of solids, for example the cleaning substance, while the viscosity at high mechanical load, i.e. at exit of the composition from a dispenser device, should be low in order to be able to exit properly from the dispenser device. As soon as the composition is on the mouth insert, however, the higher viscosity should again be set in order to prevent a drain. In addition, the pyrogenic silicate(s) can be used as dispersing aid.

The optionally in combination with the or the precipitated silicate(s) in the composition comprises pyrogenic silicate or the also optional mixture of two or more pyrogenic silicates is included in the composition preferably with a proportion of 0.1 to 2 wt.-%, based on the total composition, in the inventive composition. Preferably, the proportion of des is at least one pyrogenic silicate 0.2 to 1.5 wt.-%, even more preferably 0.2 to 1.0 wt.-%, each based on the total composition.

According to a further preferred embodiment of the composition according to the invention, the composition does not contain polyethylene glycol or no polyethyleneglycol or the proportion of polyethylene glycol or polyethylene glycols in the composition is a maximum of 0.8 wt.-%, preferably a maximum of 0.4 wt.-%. Polyethylene glycols are according to this embodiment of the invention thus comprises with a proportion of 0 to 0.8 wt.-%, preferably with a proportion of 0 to 0.4 wt.-%. The proportion is highly preferred, apart from unavoidable possible impurities and within the usual measuring accuracy, 0 wt.-%.

Polyethylene glycols can perform different functions in the composition depending on the molecular weight. Polyethylene glycols, in particular those with molecular weights of 1. 500 to 1,000,000 g/mol, can act as viscosity amplifier, but polyethylene glycols with a smaller molecular weight can also be used as a humectant. The above proportions shall apply regardless of the molecular weight of the polyethylene glycols.

According to a further preferred embodiment of the composition according to the invention—it comprises as viscosity amplifier Xanthan Gum or the viscosity amplifier Xanthan Gum. Xanthan Gum, also known as Xanthan rubber, is a high molecular weight hetero polysaccharide formed by the microorganism Xanthomonas campestris or produced from hydrolyzed starch. Essentially, Xanthan Gum consists of beta-1,4-linked glucose units with side chains containing mannose and glucuronic acid. In addition to being able to form viscous solutions beyond its suitability, Xanthan Gum has surprisingly turned out to be particularly advantageous viscosity amplifier in the compositions of the invention because of its dispersions stabilizing effect and its thixotrophic properties. The Xanthan Gum used may be of natural or synthetic origin.

With the proviso that that the viscosity amplifier component of the composition of the invention comprises Xanthan Gum, it is particularly preferred if the viscosity amplifier component comprises a mixture of Xanthan Gum and acrylates and/or polyacrylates, in particular crosslinked polyacrylates, C10-30-alkyl-acrylate cross polymer, or consists of this mixture. It is particularly preferred in both cases when the weight ratio of Xanthan Gum to acrylate(s) and/or polyacrylate(s), in particular cross-linked polyacrylates, C10-30-alkyl acrylate cross polymer, is in a range of 10:1 to 1:10, even more preferably in the range of 8:1 to 1:8, and most preferably in the range of 6.5:1 to 1:6.5.

Acrylates and/or polyacrylates, in particular crosslinked polyacrylates, C10-30-alkyl-acrylate cross polymers are particularly suitable for surfactant systems that are difficult to thicken and provide stable viscosities over a wide pH range. In addition, they have a positive influence on the foam properties.

Due to the advantageous suitability of Xanthan Gum and the acrylates and/or polyacrylates, in particular of C10-30-alkyl-acrylate cross polymers, it is therefore also provided according to the invention that the composition does not contain cellulose and/or cellulose derivatives and/or polysaccharides from algae, for example carrageenan and/or alginate, so is free of one or more of these components.

The cellulose, if present, may be present in non-derivative form and water insoluble. A cellulose is in the sense of the present invention water insoluble if dissolved in 100 g of a saturated aqueous solution at 20° C. less than 1 wt.-% of the cellulose. Non-derivative celluloses in the sense of the invention are those whose hydroxyl groups are not substituted by another chemical group. Cellulose derivatives include hydroxyalkyl celluloses, such as hydroxyethyl cellulose, carboxymethyl cellulose, hydroxyethyl cellulose, methylhydroxypropyl-cellulose and their salts, for example sodium salts. Carrageenan, also known as carrageenan, is a collective name for long-chain carbohydrates found in red algae.

In the sense of the present invention, the formulation “free of cellulose and/or cellulose derivatives” means that the composition has cellulose, cellulose derivatives or a mixture of cellulose and one or more cellulose derivatives with a maximum content of less than 0.01 wt.-%, based on the total composition without propellant gas. The proportion of cellulose, cellulose derivatives or a mixture of cellulose and one or more cellulose derivatives is thus in the range of 0 wt.-% to less than 0.01 wt.-%, based on the total composition without propellant. Preferably, the maximum permissible maximum is 0.008 wt.-%, even more preferably 0.006 wt.-% and even further preferably 0.004 wt.-%, each based on the total composition without propellant. Highly preferred is the proportion of cellulose, cellulose derivatives or a mixture of cellulose and one or more cellulose derivatives in the composition 0 wt.-%. Any unavoidable impurities should be disregarded. The usual measurement accuracy also applies.

The same applies to the formulation “free of polysaccharides from algae, for example carrageenan and/or alginate”. Corresponding composition contain carrageenan and/or alginate and/or other polysaccharides of algae, not exceeding 0.01 wt.-%, based on the total composition without propellant gas. The proportion of carrageenan and/or alginate and/or other polysaccharides from algae is thus in the range of 0 wt.-% to less than 0.01 wt.-%, based on the total composition without propellants. Preferably, the maximum permissible maximum is 0.008 wt.-%, even more preferably 0.006 wt.-% and even further preferably 0.004 wt.-%, each based on the total composition without propellant. Highly preferred is the proportion of carrageenan and/or alginate and/or other polysaccharides from algae in the composition 0 wt.-%. Any unavoidable impurities should be disregarded. The usual measurement accuracy also applies.

Furthermore, it is preferred according to the invention that the dental care and tooth cleaning-composition comprises at least one active substance or substance of content. The at least one active substance is, for example, selected from the group, consisting of hydroxyapatite or other remineralizing or ceramising substances, a fluoride active substance, a gum care agent, a disinfecting component, a means for the reduction of plaques, an anti-inflammatory agent, a preservative and any mixture thereof.

The proportion of at least one or the other active substances in the composition of the invention is 0.5 to 8 wt.-%, based on the total composition. Preferably, the proportion of at least one or the active substances is 1.5 or 2.0 to 7.5 wt.-%, even more preferably 2.5 to 7 wt.-%, each based on the total composition.

As a fluoride active substance, an amine fluoride, sodium fluoride or tin fluoride or a mixture thereof is particularly preferably used. The fluoride active substance or the fluoride active substances are preferably containing a proportion of 0.05 to 1.5 wt.-%, based on the total composition. Even more preferably, the proportion is 0.1 to 1.2 wt.-%, most preferred is the proportion in the range of 0.2 to 1.0 wt.-%.

As an alternative to the fluoride active substance, the composition of the invention may contain hydroxyapatite. In a possible embodiment of the invention, the dental care composition contains either fluorides, as a further active substance, or hydroxyapatite or other remineralizing substances and combinations of hydroxylapatite and one or more other remineralizing substances.

As a gum care agent is particularly preferred allantoin, panthenol, extracts of arnica, myrrh, ratanhia, sage, echinacea, chamomile, rosemary oil, thyme oil or aloe or any mixture of these substances used. The agent for gum care, if present, is preferably containing a proportion of 0.1 to 3 wt.-%, even more preferably with a proportion of 0.15 to 2 wt.-%, based on the overall composition.

As a means for the reduction of dental plaques, for example, chlorhexidine digluconate, zinc aspartate or arginine or any mixture of these can be used.

The agent for the reduction of dental plaques is contained, if present, preferably with a proportion of 0.2 to 3 wt.-%, even more preferably with a proportion of 0.5 to 2 wt.-%, based on the total composition.

Other active substance also include anti-inflammatory agents and disinfecting components, such as bactericides. They are, if present, preferably with a proportion of 0.1 to 3 wt.-%, even more preferably with a proportion of 0.2 to 2 wt.-%, based on the total composition contained.

It is further preferred according to the invention that the composition comprises usual excipients and additives selected from the group, consisting of flavoring agents, sweeteners, preserving agents, pigments, dyes, buffer substances and any mixture thereof. Thus, the composition of the invention may comprise, for example, one or more color pigments. Pharmaceutically and/or cosmetically acceptable color pigments are known to the skilled person. These can be used individually or in any mixtures thereof. Preferably, chlorophylline (C.I. 75810), optionally also used in mixture with other color pigments, is used as a color pigment. The proportion of color pigment, be it a color pigment or a mixture of several color pigments, is preferably 0 to 1.0 wt.-%, preferably 0.2 to 1.0 wt.-%, based on the total composition. Even more preferably, the range is 0.3 to 0.8 wt.-%.

The composition of the invention may further comprise one or more flavoring substances. Particularly preferred, the flavoring substance comprises anethole, menthol, green mint (“Spearmint”), eucalyptus or limes or any combination of these preferred flavorings. The proportion of flavoring substances, be it a flavoring substance or a mixture of several flavoring substances, is preferably 0 to 1.5 wt.-%, preferably 0.1 to 1.3 wt.-%, based on the total composition. Even more preferred the proportion is 0.2 to 1.1 wt.-%.

As further optional components, the composition according to the invention can comprise—at least one binder, at least one solvent, at least one wetting agent, at least one sweetener, at least a bleaching agent and/or at least one pH regulator. The composition of the invention may comprise as an optional component—also one or more further stabilizers, such as sodium gluconate (INCI: sodium gluconate). This at least one further stabilizer can prevent a demixing of the emulsion and/or prevent a degradation of the other components of the composition, for example the active substances. Sodium-gluconate, for example, not only prevents a demixing of the composition according to the invention, it also prevents an oxidative decomposition of the other components, in particular the active substances of the composition. These optional components may be individually or in any combinations with each other in the composition of the invention.

The active substances and additives of the composition for oral and/or tooth cleaning and dental care can basically be the ones, as are also used for already known tooth cleaning agents, such as toothpastes, tooth cleaning gels and the like. Nevertheless, certain substances of content and excipients or additives and/or their combination with each other have turned out to be particularly advantageous—to provide a composition that can be applied evenly, sparingly and simply to the mouth insert or mouth inserts of the toothbrush for simultaneous cleaning of several, preferably all teeth of a user. In addition, the composition ensures that sufficient active substance is applied, but at the same time an overdose is avoided.

All components of the composition are in a degree of purity that is suitable and necessary for dental care compositions. If no information is given hereon for certain excipients or additives on their percentage by weight, this means that the skilled person knows the preferred ranges or can simply determine them.

The pH of the dental care and tooth cleaning composition according to the invention is preferably in the range of 5.5 to 9.0, even more preferably in the range of 6.0 to 8.0 and most preferred in the range of 6.5 to 7.5.

Also included to the extent of the present invention are such compositions which, with the exception of usual impurities, consist only of the above-mentioned essential, and optionally one or more of the optional constituents.

The indication wt.-%, as used in the present patent application, means, unless otherwise stated, that the proportion is related to the total weight of the composition without propellants.

A particularly preferred composition according to the invention comprises:
20 to 50 wt.-%, preferably 25 to 40 wt.-%, hydrogenated starch hydrolysate, which preferably has a sorbitol content of65 to 75 wt.-%,
15 to 30 wt.-%, glycerin,
1 to 2 wt.-% aminfluoride-containing raw material with 20-50% active substance content.
2 to 20 wt.-%, preferably 6 to 15 wt. %, precipitated silicate of category 1, preferably Zeodent 103,
1 to 10 wt.-%, preferably 3 to 7 wt.-%, precipitated silicate of category 2, preferably Zeodent 115,
1 to 10 wt.-%, preferably 1 to 3 wt.-%, precipitated silicate of category 3, preferably Zeodent 167,
0.1 to 2 wt.-%, preferably 0.2 to 1 wt.-%, sodium lauryl sulfate,
0.1 to 2 wt.-%, preferably 0.15 to 1 wt.-%, cocamidopropyl betaine,
0.05 to 0.2 wt.-% Xanthan Gum,
and at least 10 wt.-% water ad 100 wt.-%.

Another particularly preferred composition of the invention comprises:
16.25 to 30 wt.-% sorbitol,
15 to 30 wt.-% glycerin,
1 to 2 wt.-% aminfluoride-containing raw material with 20-50% active substance content,
6 to 13 wt. %, preferably 7 to 13 wt.-%, precipitated silicate of category 1, preferably Zeodent 103,
3 to 7 wt.-% precipitated silicate of category 2, preferably Zeodent 115,
1 to 3.5 wt.-%, preferably 1.5 to 3 wt.-%, precipitated silicate of category 3, preferably Zeodent 167,
0.1 to 2 wt.-%, preferably 0.2 to 1 wt.-%, sodium lauryl sulfate,
0.1 to 2 wt.-%, preferably 0.15 to 1 wt.-%, cocamidopropyl betaine,
0.05 to 0.2 wt.-% Xanthan Gum,
and at least 10 wt.-% water.

In these two particularly preferred composition according to the invention, the proportions of the precipitated silicates, the glycerin, the surfactants, the viscosity amplifier is each selected so that the above disclosed wt.-% proportions of 10 to 23.5 wt.-% precipitated silicate, 15 to 30 wt.-% glycerin, 0.2 to 4.0 wt.-% surfactant and 0.05 to 0.5 wt.-% viscosity amplifier and at least 10 wt.-% water are maintained. In one embodiment of the present invention, the basic formulation of the composition consists of the components mentioned in the preceding section with the specified proportions. This basic formulation may provide for the finished composition without propellant gas complementary to these components at least one active substance, for example selected from the group consisting of hydroxyapatite or other remineralizing or ceramising substances, a fluoride active substance, a gum care agent, a disinfecting component, a means for the reduction of dental plaques, an anti-inflammatory agent, a preserving agent and any mixture thereof, and/or at least common excipients and additives selected from the group consisting of flavorings, sweeteners, preserving agents, pigments, dyes, buffer substances and any mixture thereof, in the proportions specified above, as well as a means of adjusting the pH value.

This particularly preferred composition may either, apart from usual impurities, consist only of these constituents, or it may further include 0.3 to 1.0 wt.-% chlorophylline and/or 0.5 to 1.5 wt % anethole or menthol or limonene or any mixture thereof. In addition, in the composition may still comprise saccharin or another sweetener, a pH regulator, PEG-hydrogenated castor oil (INCI: PEG-40 Hydrogenated Castor Oil) and/or sodium gluconate (INCI: Sodium Gluconate). According to a further embodiment according to the invention, the composition, apart from usual impurities, consists only of the said essential and one or more of the said optional components.

When using the composition of the invention, it is preferably provided according to the invention that the composition of the invention is transferred from the foamable mixture in a second stage in a foam, preferably in a foam spray, with a certain lifetime or into an aerosol. Therefore, the surfactants and/or the viscosity-amplifiers in the composition have a special importance. With regard to particularly preferred substances and combinations thereof, reference is made to the above disclosure, in which these substances are discussed in detail.

According to a further aspect, the present invention thus relates to the above described in detail dental care and tooth cleaning composition in combination with a propellant gas contained in the composition or a propellant gas mixture. Preferably, the propellant or propellant mixture in the composition of the invention is contained in a quantity of at least 0.2 wt.-%, even more preferably of at least 0.5 wt.-%, even more preferably of at least 1 wt.-%. Furthermore, it is preferred that the content of propellant gas or propellant mixture in the composition is not higher than 15 wt.-% and even more preferably not higher than 13 wt.-%, since otherwise the agent would form a too voluminous foam during application and use. The propellant or propellant mixture thus acts as a foaming agent. It is most preferred that the content of propellant or propellant-medium mixture in the composition is in a range of 2.5 to 12 wt.-%.

The present invention thus relates in a further aspect also an aerosol or a foam, which is formed from the composition of the invention and which can be evenly distributed on the mouth insert or the mouth inserts. The aerosol or foam formed is stable for a sufficient period of time and thus adheres to the mouth inserts for a sufficient period of time. A draining of the composition or the resulting foam or the foam drops or the aerosol-drip before insertion of the mouth insert or the mouth inserts into the mouth can thus be avoided.

Aerosols and foams offer the advantage of long durability, precise dosage and economical use. In addition, no microorganisms can enter the dispenser, so that even unpreserved products are possible.

An aerosol is a heterogeneous mixture of non-gaseous substances in a gas. It is generated only by actuating the outlet valve of a suitable dispenser—and storage device for the inventive tooth cleaning and dental care composition and is thus produced only if necessary and in the required quantity. The foam is the final product, which is formed by the dispenser and storage device from the composition of the invention. A foam includes gaseous bubbles enclosed by solid or liquid walls. The composition contained in the dispenser-device should contain tensides or similar crosslinking agents, as well as preferably foam stabilizers, which allow a sufficiently stable foam production. Depending on the special properties of the surfactants contained in the composition, foam bubbles of different sizes, wall thickness and service life are produced. Basically, the durability of liquid foams is limited. This may depend on the viscosity of the liquid forming the foam, i.e. the composition described herein, and/or the substances contained in the composition.

The aerosol leaking out of the dispenser or the foam exiting from the dispenser then expands further and can thus fill the recesses more or less completely, provided for the teeth, described herein also as U-shaped tubs, in the mouth inserts. This ensures that the active substances are evenly distributed and that all teeth can be cleaned and treated efficiently. At the same time, the aerosol or foam offers the advantage that the composition can be applied sparingly and an overdose can be avoided.

According to a further aspect, the present invention relates to a system, which comprises the herein described dental care and tooth cleaning composition, and a container or dispenser with means for stocking and dispensing the dental care and tooth cleaning composition. The dental care and tooth cleaning composition can be mixed with propellant or propellant gas mixture.

In order for such systems to produce and release a suitable aerosol or a suitable foam, other technical parameters in addition to the composition, such as the ratio of active substance solution to propellant gas, the type of propellant gas, the size of the valve opening and/or the size of the spray head opening play a role. The size of the valve opening and the size of the spray head opening are directly correlated with the droplet size of the aerosol or foam. For the valve head it is preferably between 0.2 mm and 4 mm, for the spray head preferably between 0.4 mm and 6 mm.

According to the invention, the dispenser comprises a means for stocking the composition of the invention, a conveying system for conveying the composition and an outlet valve for outputting the composition. The conveying system preferably conveys the composition to the outside by means of a propellant gas, compressed air or via a pump system. The dispenser is sufficiently pressure-stable compared to for example the expansion pressure of the propellant gas-containing composition or the propellant gas. The composition and the dispenser work together in such a way that when actuating the outlet valve, the propellant gas-containing tooth cleaning agent is exiting in a strand-shaped, little foamed form from the outlet valve and is further foamed or expanded temporally delayed at the application site.

In an embodiment according to the invention, the conveying system conveys the composition by means of a propellant gas or propellant mixture, compressed air or via a pump system from the dispenser. The outlet opening of the dispenser, for example, the foam-nozzle, is preferably designed in such a way that an even, precise and economically distribution of the composition according to the invention on the mouth insert or the mouth inserts is ensured. The outlet valve is preferably manually actuated.

According to a preferred embodiment, the dispenser is an aerosol can or spray can. This is preferably a metallic container or preferably a plastic container, which contains the composition of the invention and is under pressure due to the use of a propellant gas or propellant mixture. Due to the internal pressure of the spray can, the contents are released exactly when the outlet valve is operated. A part of the propellant gas or propellant mixture is dissolved in the active agent solution, i.e. the actual composition of the present invention, before and the other part of the propellant gas or propellant mixture is present as gas. If the outlet valve is now actuated, the gaseous portion of the propellant gas or propellant mixture drives the active substance solution through the valve outwards. Now the propellant gas or propellant mixture contained in the composition evaporates extremely quickly and the remaining active substance solution is distributed very finely and evenly under the formation of a foam. As a propellant gas for an aerosol can be preferably air, carbon dioxide, initrous oxide (N₂O), n-propane, n-butane, n-pentane, isopropanol, isobutene or any mixture thereof. The amount of propellant gas or propellant mixture in the dispenser is chosen in such a way that it can be ensured that the can be completely emptied and that there is always enough propellant gas or propellant mixture gas in liquid form in the composition until the can is emptied and can act as a foaming agent.

According to an alternative preferred embodiment, the dispenser is a bag-on-valve can. With this aerosol technology, even thicker products can be carried out. Here, propellant gas or propellant mixture and active substance solution. i.e. the above described dental care and tooth cleaning composition, are separated from each other and are present in two separate chambers. A bag, for example an aluminum bag or a laminated aluminum bag, contains the active substance solution and this bag is connected to the valve. A propellant gas or propellant gas mixture, for example compressed air, nitrogen, carbon dioxide, nitrous oxide (N₂O), n-propane, n-butane, n-pentane, isopropane, isobutene, isopentenyl, a propane-butane mixture or a butane-pentane mixture, surrounds the bag and provides the necessary pressure to convey the composition from the dispenser. When actuating the outlet valve, the propellant gas now squeezes product out of the bag. The outer can is preferably made of tinplate, aluminum or polyethylene-terephthalate, because these materials are stable, light and very recyclable. It is preferred according to the invention that the bag-on-valve can for stocking the dental care and tooth cleaning composition according to the invention contains a single bag with the finished composition and part-mixtures are not stored in separate bags and are mixed together only when actuating the discharge valve.

According to a particularly preferred embodiment of a bag-on-valve can or a bag-on-valve system is also located in the composition of the invention-a small proportion of propellant gas or a propellant gas mixture thereof. This proportion is preferably in the range of 0 to 8 wt.-%, even more preferably in a range of 0.5 to 7 wt.-% and most preferred in a range of 1.5 to 6 wt.-%.

The propellant gas is preferably selected from the group consisting of isobutene, n-butane, isopentenyl, n-pentane, nitrous oxide (N₂O) or any mixture thereof.

In an embodiment of the invention, the propellant is nitrous oxide (laughing gas). The nitrous oxide is used in a quantity that the resulting pressure in the dispenser device is preferably at least 5 bar. Even more preferably, the pressure in the dispenser device is in the range of 6 to 10 bar.

Nitrous oxide is understood as a non-compressible liquid due to its very low boiling point in the sense of the present invention. In contrast, for example, n-butane, isobutene, n-pentane and Isopentenyl are compressible liquids.

In a further embodiment of the invention, the propellant gas mixture is a mixture of n-butane, isobutene, n-pentane and Isopentenyl. Preferably, the mixture has a proportion of isopentan of at least 70% and a proportion of isobutane of at least 20%, n-pentane and n-butane are each contained with a maximum of 5%, even more preferably of no more than 3.5%. Even more preferably, the propellant gas mixture comprises 0.1-2% n-butane, 22-28% isobutane, 0.1-3% n-pentane and 72-78% isopentan. The proportions of the four components are each selected in the mixture in such a way that a total of 100% for the propellant gas mixture is achieved. The use-concentration of such a mixture is preferably in the range of 2 to 12 wt.-%, even more preferably in the range of 3 to 10 wt.-%.

The propellant gas or the propellant gas mixture is selected or assembled according to a further preferred embodiment of the present invention so that it has at normal pressure (1.013 hPa) a boiling point of −10° C. or higher, even more preferably of −8° C. or higher. According to an embodiment of the invention, the propellant gas is n-butane. Preferably, the concentration of n-butane, based on the composition without propellant gas, so the pure liquid formulation, is in the range of 2 to 12 wt.-%, even more preferably in the range of 3 to 10 wt.-%.

The propellant gas or propellant gas-mixture, contained in the composition of the invention, acts as foaming agent.

Also for the embodiment of the aerosol can are used for the preparation of a foam or an aerosol as propellant gas preferably n-propane, n-butane, n-pentane, isopropane, isobutane, isobutane, isobutene or any mixture thereof. Alternatively, air, carbon dioxide, nitrous oxide or any mixture thereof can be used as a propellant gas. It is again particularly preferred that the propellant gas or the propellant gas mixture is selected so that it does not contain a compound that at normal pressure has a boiling point of less than −10° C., even more preferably of less than −8° C.

Also for the embodiment of the aerosol can is the propellant gas in an invention-corresponding embodiment of nitrous oxide (laughing gas). The nitrous oxide is used in a quantity that the resulting pressure in the dispenser device is preferably at least 5 bar. Even more preferably, the pressure in the dispenser device is in the range of 6 to 10 bar.

In a further embodiment according to the invention, the propellant gas mixture in the aerosol can is a mixture of n-butane, isobutene, n-pentane and Isopentenyl. Preferably, the mixture has a proportion of isopentenyl of at least 70% and a proportion of isobutane of at least 20%, n-pentane and n-butane are each contained with a maximum proportion of 5%, even more preferably of no more than 3.5%. Even more preferably, the propellant gas mixture comprises 0.1-2% n-butane, 22-28% isobutane, 0.1-3% n-pentane and 72-78% isopentenyl. The proportions of the four components are each selected in the mixture in such a way that a total of 100%/a for the propellant gas mixture is achieved. The application concentration of such a mixture is preferably in the range of 2 to 12 wt.-%, even more preferably in the range of 3 to 10 wt.-%, based on the composition without propellant gas, thus the pure liquid formulation.

The propellant gas or the propellant gas mixture of the aerosol can is set according to a further, preferred embodiment of the present invention selected or assembled—that it has at normal pressure (1,013 hpa) a boiling point of −0° C. or higher, even more preferably of −8° C. or higher. According to an embodiment of the invention, the propellant gas is n-butane. Preferably, the concentration of n-butane, based on the composition without propellant gas, so the pure liquid formulation, is in the range of 2 to 12 wt.-%, even more preferably in the range of 3 to 10 wt.-%.

An alternative to the production of a foam or an aerosol from the composition is the use of a manual or electric pumping system. When the pump is actuated, an overpressure is created between an outer and an inner container, which forms the aerosol or foam after actuation of the valve. The application of the composition as foam is particularly preferred.

In a further aspect, the present invention relates to the use of the above-described system for stockpiling and dispensing the dental care and tooth cleaning composition according to the invention. In this process, the composition is preferably applied as a little foamed strand to the mouth insert or the mouth inserts of the tooth cleaning device. After application, the strand at 20° C. and normal pressure preferably has an expansion rate of more than 100 vol.-% in 3 to 5 seconds. Even more preferably, the expansion rate is at 20° C. and normal pressure after 3 to 5 seconds in a range of 150 vol.-% to 1000 vol.-%. The thus obtained foam is then used for cleaning tooth surfaces, tooth spaces and the oral cavity.

In a further aspect, the present invention relates to a method for simultaneous cleaning of several, preferably all teeth by means of a vibration-tooth brushing device for the simultaneous cleaning of several, preferably all teeth of a user, wherein the composition is presented by applying the composition to a mouth insert or several mouth inserts of the tooth brushing device. Tooth cleaning is used in particular for the prevention and/or treatment of diseases of the teeth, dentine and/or gums.

The method according to the present disclosure accordingly includes the following steps:
Providing a dental care and tooth cleaning composition, which comprises as essential components: 10 to 40 wt.-%, preferably 15 to 30 wt.-% glycerin, 10 to 23.5 wt.-% at least one precipitated silicate, preferably 10 to 23.5 wt.-% a mixture of 2 to 4 precipitated silicates, 0.05 to 0.5 wt.-% at least one viscosity amplifier, at least one surfactant, preferably with a proportion of 0.2 to 4.0 wt.-% of one or more surfactant(s), and at least 10 wt. % water,
Coating/applying the composition to a mouth insert or several mouth inserts suitable for cleaning several, preferably all teeth of a user,
Inserting the mouth insert or mouth inserts with the composition into the user's mouth and
Cleaning several, preferably all teeth of the user with the mouth insert or mouth inserts and the composition. Cleaning is preferably carried out by the mouth insert or the mouth inserts by means of a drive device to be stimulated to vibration.

The provided dental care and tooth cleaning composition preferably has a viscosity of 200-30,000 m Pa s.

For the method according to the invention, the herein described dental care and tooth cleaning composition is particularly suitable. It not only provides a sufficient amount of active substance, but can also be distributed particularly evenly and easily on the mouth insert or the mouth inserts. It also avoids overdosage such as those that would occur with conventional toothpastes, pastes or gels.

The provided composition is thus preferably the dental care and tooth cleaning composition according to the invention, comprising
15 to 30 wt-% glycerin,
optionally at least one other humectant, in addition to glycerin, wherein the at least one other humectant preferably is or comprises sorbitol or hydrogenated starch hydrolysate, which preferably has a sorbitol content in the range of 65 to 75 wt.-%,
10 to 23.5 wt.-% at least one precipitated silicate,
0.2 to 4.0 wt.-% at least one surfactant,
0.05 to 0.5 wt.-% at least one viscosity amplifier, and
at least 10 wt.-% water.

The composition preferably has a dynamic viscosity of 200-30,000 m Pa s without propellant gas. Even more preferably, the viscosity of the composition without propellant gas is in a range of 400-10,000 m Pa s and even more preferably at less than 10,000 m Pa s. For example, a viscosity in the range of 500-9,000 m Pa s at 20° C., is even more preferably.

With regard to the preferred essential and optional content and excipients or additives as well as their preferred proportion ranges is expressly referred to the above specifications in connection with the composition as such.

Coating/application of the composition to the mouth insert or mouth inserts is preferably carried out as foam, preferably as foam spray, or as an aerosol. The composition can then expand even further on the mouth insert or mouth inserts and form a tooth cleaning mousse. Preferably, the initially slightly foamed toothpaste strand after application at 20° C. and normal pressure has an expansion rate of more than 100 vol.-% in 3 to 5 seconds. Even more preferably, the expansion rate is at 20° C. and normal pressure after 3 to 5 seconds in a range of 150 vol.-% to 1000 vol.-%.

By applying the composition to the mouth insert or mouth inserts, the uniform and economical distribution of the composition for the user is further facilitated and good cleaning and care of the teeth and gums can be ensured. In addition, the aerosol or foam (directly or as a foam spray) or the foam has a certain durability before it dissolves into its low-viscosity basic composition. During this time, the mouth insert or the mouth inserts can be inserted into the user's mouth. The foam/aerosol or mousse applied to the mouth insert is then crushed by the space between the teeth and mouth insert and evenly distributed. Thus, the foam or aerosol can be applied very sparingly to the mouth insert or the mouth inserts.

The application of the composition is preferably carried out with the help of one of the herein described dispenser by which the composition of the invention can be converted into a foam, preferably a foam spray, or an aerosol. With regard to preferred explanations for this dispenser, reference is expressly made to the above disclosure.

The tooth cleaning method of the invention further comprises the insertion of the mouth insert or the mouth inserts with the composition in the mouth of a user and the simultaneous cleaning and care of several, preferably all teeth of the user by vibration of the mouth insert. The exact embodiments of the mouth and tooth cleaning via different cleaning structures on the inner surfaces of the mouth insert, vibration frequencies, cleaning programs and application duration are not limited in the present invention.

A mouth insert for use in teeth cleaning and care according to the invention can have for cleaning all teeth two U-shaped tubs for receiving the teeth, wherein the two tubs fit the teeth of the user and are opened in opposite-set directions. The mouth insert is at least coarse, preferably individual, adapted to the denture and jaw shape of a user and dimensioned accordingly. If a too low viscous tooth cleaning composition is used, this can, when inserting the mouth insert from the lower tub-flow out or accumulate at the outer edge of the lower tub, which can negatively affect the cleaning-result, in particular on the chewing surfaces of the teeth of the lower jaw. An aerosol, foam or mousse, on the other hand, remains in the tub for a long time due to its inherent strength, even if it is turned downwards with the opening.

In a preferred embodiment of the invention, the mouth insert or the mouth inserts for cleaning the teeth, the dentine and the gums in the mouth of the user are stimulated by a motor to oscillations/vibrations with a frequency in the range of 20 to 700 Hz, preferably to 600 Hz.

The simultaneous cleaning of the several, preferably all teeth of the user, preferably takes between 2 seconds and 5 minutes, even more preferably the tooth brushing time is in a range of 3 seconds to 4 minutes. Alternatively, the upper limit of the cleaning time is 2 minutes, 1.5 minutes or 1 minute.

In an embodiment of the invention, the mouth insert has a shape that surrounds the teeth of a user during use, so that during the cleaning process chewing surfaces and inner and outer flanks of the teeth are each opposite to a surface of the mouth insert, and wherein a variety of cleaning structures on the teeth facing sides of the mouth insert are provided.

In an embodiment according to the invention, the mouth insert is individually adapted to the dental form of the user. This adjustment can be done by conventional negative positive dental impressions, scanning and subsequent 3D printing of the mouth insert, or other suitable procedures. Preferred materials for oral use are biocompatible plastics, e.g. biocompatible polyamide.

In an embodiment according to the invention, the mouth insert is one-piece or, in each case for the upper jaw and the lower jaw, formed two-piece.

FIG. 1 shows a possible embodiment of a mouth insert for use with a composition according to a preferred embodiment of the present disclosure. In particular, FIG. 1 shows a perspective view of a one-piece mouth insert 10. The invisible lower structure of the mouth insert is substantially identical to the upper one, wherein adaptation-related shape deviations are possible. The wall thickness of walls 12 of the mouth insert 10 is typically 1 to 2.5 mm. The outer boundary of the walls 12 usually runs 1 to 3 mm above the gum edge. The distance between the cleaning surfaces, i.e. those surfaces of the mouth insert 10, which are facing the tooth surfaces of the user during the cleaning process, and the tooth surfaces is 0 to 5 mm depending on the selected cleaning structures 14. In a preferred embodiment, cleaning structures 14 are provided on the cleaning surfaces.

The mouth insert 10 of the embodiment in FIG. 1 comprises an attached coupling element or a coupling portion 20 for connecting the mouth insert 10 with a drive device (not shown). The drive device is basically comparable with drive devices as they are known from the prior art in the field of sound toothbrushes or the like. The coupling with the drive-device is carried out via a suitable bore 26.

FIG. 1 shows that a mouth insert that is relatively precisely adapted to the user's teeth has many recesses (corresponding to the teeth) and webs (corresponding to the tooth spaces and chewing surfaces). These bumps make it much more difficult to use with a conventional toothpaste or similar preparations. Either a lot of toothpaste has to be applied, which increases application costs and wastes resources. An accurate distribution of a viscous tooth-cream with a brush or a spatula would also be very time consuming for the user. The cleaning structures 14 (shown in FIG. 2 and described in detail below) additionally produce fine unevenness, which can hardly be reached or filled with a conventional toothpaste.

FIG. 2 shows a detailed view of cleaning structures 14 in the form of cleaning elements, which can be provided on the surface of the mouth insert 10 of FIG. 1 in the areas that are introduced into the oral cavity and in particular in the recesses that come into contact with the teeth. In this case, they are each cleaning elements, which are formed one piece with the mouth insert 10 on its surface. In the area of the chewing surfaces, diamond-shaped cleaning elements 32 are provided in this embodiment, in the area of the front and rear tooth surfaces are each formed cylindrical cleaning elements 30. Due to the diamond shape, the cleaning elements 32 are harder than the cylindrical cleaning elements 30, so that a more intensive tooth brushing effect is achieved in the area of the chewing surfaces. Of course, the cleaning elements 30 on the front and rear tooth surfaces can be differently formed. Likewise, in each of the areas shown in FIG. 2, i.e. in the area of the posterior tooth surfaces, the chewing surfaces and the front tooth surfaces, other cleaning structures 14 could be provided, for example rubber coating layers and/or strip brushes.

In FIGS. 3 to 5, further embodiments of a mouth insert or parts thereof are shown, which are particularly suitable for use in the method according to the invention or for use according to the invention. The mouth insert may be designed according to an inventive embodiment of the method or the inventive use also so that it comprises a support structure and a silicone shell, which essentially completely surrounds the support structure.

The task of the support structure is to maintain the tension of the mouth insert and, above all, to transmit vibrations from a drive device to the teeth of the user. FIG. 3 shows a vertical longitudinal cross-sectional view of a toothbrush device 100 that can be applied according to the invention. The support structure comprises at least one mouth insert element 30a for the upper jaw of the user and at least one mouth insert element 30b for the lower jaw of the user. The mouth insert element for the upper or the lower jaw is preferably adapted for all teeth of the upper or of the lower jaw. However, the mouth insert elements can also be multi-part formed, e.g. each a section for the right and the left denture half of the respective jaw or each a section for the tooth inner, tooth outer and chewing flanks. However, the embodiment with exactly one mouth insert or mouth insert element for the upper and lower jaw is preferred. The mouth inserts preferably comprise a stable base holder (hereinafter also referred to as base body), which function for vibration coupling with a drive device 10. This basic body essentially comprises a biocompatible material that preferably meets EN ISO 10993-1 and/or is certified for food contact in accordance with EU Plastics Directive 2002/72/EC. Particularly preferred is the use of biocompatible polyamide (homo- and/or copolyamide), such as PA6, PA12, PA11, or, particularly preferred, PA6.6. In another embodiment is used for the production of the base body of the mouth inserts polyamide 12. e.g. in the version PA 2200 (white) or PA 2201 (transparent). The entire base body may be made of the biocompatible material as described above.

The mouth insert element for the upper jaw of the user and the mouth insert element for the lower jaw may be connected to each other at their ends, which are opposite of the coupling portion 20. This refers to the portions of the mouth insert elements, which are intended for cleaning the rear side teeth.

The mouth insert elements may be designed in such a way that they each have a tray-shaped or U-shaped cross-section formed by a support structure floor, which essentially corresponds to the shape of the bite surface of a human denture (transverse plane of the human body), an outer wall and an inner wall. The support structure may be made of homo- or copolyamide, preferably food-safe homo- or copolyamide, further preferably from PA 6, PA, 6.6, PA 4.6, PA 11, PA 12, PA 1010, PA 610, copolyamides or polyamide mixtures thereof, and particularly preferably PA 6.6, copolyamides or polyamide mixtures.

The support structure comprises in a preferred form in addition a coupling portion 20, which has at least two arms 22 and a drive connection portion 24, wherein the at least two mouth insert elements 30a and 30b are each connected to an arm 22 of the coupling portion 20 and the drive device-connection portion 24 is adapted to be connected to the drive device 10.

In other preferred forms, the oral elements can also be connected by two or more arms to the drive connection portion 24. The tooth brush device 100 is characterized in an embodiment in that the arms 22 of the coupling portion 20, which are connected to the mouth insert elements 30a and 30b, each have at least one spring portion 22a or 22b. The spring portions 22a and 22b preferably have a lower stiffness than the remaining areas of the arms 22 or the arm areas without spring portions, and a high flexibility, so that the spring portions bias the mouth insert elements 30a and 30b at load in the direction of a basic position. The spring sections 22a and 22b allow on the one hand an easier insertion of the mouth elements into the mouth. In addition, self-resonance vibrations of the mouth insert elements 30a and 30b are attenuated to each other by the spring coupling portion 20. It should be noted that the spring portions 22a and 22b in some embodiments can extend over the total length of an arm 22. Preferably, the spring portions extend more than 20% to 100% of the length of an arm 22 and in particular more than 35% to 95% or more preferably 65% to 90% of the arm 22. The spring portions 22a and 22b of the coupling portion 20 continue to result in improved vibration transmission to the user's teeth.

The coupling portion 20 can be formed with the spring portions in one piece. Likewise, the spring portions can be formed one-piece with the mouth insert elements and/or the non-spring loaded portions of the respective arm. The single-piece embodiment improves the vibration transmission between drive device 10 and mouthpiece. In addition, spaces are avoided, which could increase the cleaning effort or worsen the hygienic conditions of the device. It is preferred that the coupling portion 20 for better control of the self-resonance-behavior consists of the identical material, preferably biocompatible polyamide, particularly preferably PA 6.6 or PA 12 as PA 2200 or PA 2201, as the base parts of the mouth inserts.

Support structures can also be used, in which the spring portions are interchangeable via plug-in or screw connections. Such a modular design of the coupling portion 20 can enable maintenance, which can make the replacement of the entire coupling portion 20 unnecessary.

The spring portions can be bent up and down in comparison to the respective arm of the coupling section 20 to which they are connected, tapered and curved or S-shaped up and down. The tapering of the coupling portions can be caused by flat-pressing (e.g. be formed under heat and pressure or rollers) of the respective arms of the coupling portion, and the S-shape can be formed by subsequent up and down the flat pressed arms, in the plane that runs lengthwise through the arms 22 and the drive connection portion 24 of the coupling portion 20, that is, in the vertical symmetry plane of the denture of the user.

Also, the spring portions can be formed by several notches on the outer sides and/or their inner sides each on the arms of the coupling portion. Preferably, the spring portions by incisions on the outer sides of the arms of the coupling portion or alternating and offset to each other at the outer and inner sides of the coupling portion are formed, which are perpendicular to the longitudinal axis of the respective arm and are cut at least up to half of the respective arm. In a particularly preferred embodiment, however, the support structure is manufactured directly by injection molding or vacuum injection molding, so that a post-treatment by incisions or heating and flat pressures is possible, but not necessary. Instead, the shape of the spring portions can be determined directly by appropriate formation of the mold.

The longitudinal cross-section through the three arms may preferably have Y-shape. Alternative cross-sections are tuning fork shape or, without the third arm, U-shape or horseshoe shape. Instead of the third arm, the drive clutch section 24 may also be provided directly at the point where the two arms 22 meet, which are connected to the mouthpieces.

In FIG. 4A a vertical longitudinal cross-section view of a preferred coupling portion 20 is shown. The coupling portion 20 has two arms 22, each comprising a spring portion. The spring portions are formed by several tray-shaped incisions 28 on the outer flanks of the arms 22 of the coupling portion 20. The edges of the incisions are preferably rounded. Alternative incision-shape (V-shaped, U-shaped etc.) are also possible.

Preferably, the coupling portion 20 for better control of the own-resonance behavior of the same material, as the support structures or the base body of the mouth insert elements 30a, 30b.

FIG. 4B shows another design form of a coupling portion 20. Here the arms 22 are provided with spring-loaded portions 29, which are formed as in the vertical plane up and down curved meanders. Other formations of the meanders, e.g. in the horizontal plane (transverse plane of the human body or perpendicular to the represented image plane) or as helix or spiral curved elements, are possible, but not explicitly represented. The spring portions are preferably tapered in comparison to the arms 22 without spring portions. In one embodiment, the strength of the arms in the range of the spring portions is 10 to 50%, preferably 20 to 40% of the strength of the arms without the spring portions.

The drive device connection portion 24 may optionally also comprise spring portions or, as shown in FIG. 4B, by a spring portion extending between the drive device and the connection point of the arms 22, are provided. Here these are provided at the same positions of the top and the bottom of the drive connection portion 24. Corresponding spring portions can also be provided as the spring sections 22a, 22b.

FIG. 4C shows another embodiment of a coupling portion 20. Here, the arms 22 and the drive connection portion 24 are provided with the same spring pattern, which is formed by incisions 28 alternating at the top and bottom of the arms 22 and the drive connection portion 24. The incisions can be V-shaped or have a different shape, as shown in FIG. 4C. FIG. 4D shows another embodiment of a coupling portion 20. Here the arms 22, similar to FIG. 3B, formed by up and down bending the tapered arms 22. However, the drive device connection portion 24 is not provided as an additional arm, but as a simple means of connection, for example as an opening, hole or pin, at the position at which the arms 22 converge. This embodiment of the drive connection portion 24 is fully compatible with the formations of the arms according to FIG. 4A to 4C.

FIG. 5 shows an r modification of the first preferred embodiment of the mouth insert. In this case, at least one of the mouth insert elements 30a or 30b comprises at least a first portion and a second portion, which are connected to a spring portion or pivot spring portion 130a or 130b. The pivot spring portion lies in the denture plane (transverse plane of the human body), which extends essentially parallel to and between the mouth insert elements 30a and 30b, has a lower stiffness than the first and second portions to facilitate a deformation of the respective mouth insert element 30a or 30b in the denture plane. The bite plane is defined as the plane corresponding to the chewing surfaces of the teeth of the user corresponding sides of the mouth insert elements 30a and 30b.

In this embodiment, the outer walls 11a each have in the area of the pivot-spring portions 130a and 130b a recess, which is bridged by vibration coupling-mechanism. The vibration coupling mechanisms optionally comprise a first coupling portion and a second coupling portion, which are arranged on opposite sides of the recess of the outer wall, extend to each other and touch each other.

The mouth insert 30a for the upper jaw of the user and the mouth insert-element for the lower jaw 30b can be connected at their ends 120, which lie opposite the coupling portion 20.

According to this embodiment, the support structure may also comprise a mouth insert for the upper jaw of the user and a mouth insert for the lower jaw of the user and a coupling portion, which has two arms and a drive-device connection portion 24, wherein the mouth insert elements are each connected to one arm of the coupling portion and the drive device-connection portion is adapted to be connected to a drive device above its support structure. This embodiment differs from the first embodiment in that that the support structure has only optional arms with spring portions and instead comprises at least one of the mouth insert elements at least a first portion and a second portion, which are connected to a pivot spring portion, which has a lower stiffness in the denture plane, which extends substantially parallel to and between the mouth inserts, than the first and second portion to facilitate a deformation of the respective mouth insert-elements in the denture plane. In other words, the arms of the coupling portion of the support structure of the second embodiment need not be equipped with the respective at least one spring portion. However, it is expressly advantageous and therefore desirable that the first and the second embodiment are combined with each other.

Accordingly, the support structure of the second embodiment may also have such arms of the coupling portion 20, which are connected to the mouth insert elements and which are each provided with at least one spring portion. The spring-strokes of the spring portions of the arms 22 of the support structure of the first preferred embodiment (in the sagittal plane of the human body) are substantially perpendicular to the spring-stroke of the pivot spring portion 130a, 130b of the second preferred embodiment (in the transverse plane of the human body).

In the support structure of a preferred shape, the mouth insert elements each comprise a base body of biocompatible material, preferably biocompatible polyamide, which is roughly adapted to a shape of the teeth of the lower jaw or upper jaw of a user. The base body is responsible for the transmission of the vibrations towards the cleaning surfaces and consists in an embodiment entirely of a biocompatible polyamide. Particularly preferred is the use of biocompatible polyamide, such as PA6, PA6.6, PA11, or PA12, preferably PA 6.6. In one embodiment issued for the production of the base body of the mouth insert elements polyamide 12, e.g. in the embodiment PA 2200 (white) or PA 2201 (transparent). Additives, dyes, and/or reinforcing fibers can optionally be added to the polyamide.

In addition, the mouth insert elements may include an insert body made of a thermoplastic material, i.e. to the sides applied to the teeth of the user during the use of the device, fixed (e.g. via gluing, impressions or a kind of Velcro connection) insert body made of a thermoplastic material, which is precisely adapted to a form of the teeth of the lower jaw or upper jaw of a user. As a thermoplastic material, a thermoplastic elastomer is used in one embodiment. Preferred are polycaprolactone (PCL), e.g. Polydoh® of the company Polymorph, thermoplastic polyester elastomer TPE-E, e.g. Keyflex® of the company LG Chemie, thermoplastic elastomer based on urethane-TPE-U e.g. Elastollan® (BASF), or mixtures of these substances. Additives or reinforcing fibers can be optionally added to these materials.

The support structure can be designed in such a way that the insert body each have a variety of cleaning structures on their inner surfaces, i.e. cleaning surfaces facing the teeth of the user. The cleaning structures may include rubber coatings mounted on the inner surfaces of the insert body, cleaning elements that are formed one piece with surface areas of the insert and/or strip brushes, which are mounted on the inner surfaces of the insert body. Examples and more detailed descriptions of the cleaning structures and their implementation are described in DE 102015109891 A1, FIG. 4 and in their paragraph [0050]. Thus (with reference to FIG. 4 of DE 102015109891 A1, wherein this figure is included herein with reference) the cleaning structures can be formed in the form of cleaning elements, which may be formed on the surface of the mouth insert, whose silicone shell or the surfaces of the mouth insert elements can be provided. The cleaning elements can be formed one piece with the mouth insert on its surface or one piece with the silicone sleeve. In the area of the chewing surfaces, diamond-shaped cleaning elements are provided in this embodiment, in the area of the front and rear tooth surfaces each cylindrical cleaning elements. Due to the diamond shape, the cleaning elements are harder than the cylindrical cleaning elements, so that a more intensive tooth brushing effect is achieved in the area of the chewing surfaces. Of course, the cleaning elements on the front and rear tooth surfaces can be differently formed. Likewise, in each of the shown areas, i.e. in the area of the posterior tooth surfaces, the chewing surfaces and the front tooth surfaces, other cleaning structures could be provided, for example rubber coating layers and/or strips-brush.

In the support structures described above, the mouth insert elements are optionally adapted for cleaning the teeth of the entire upper jaw or the entire lower jaw of the user.

In particular, a varying shape of the cleaning structures makes the use of conventional toothpastes even more difficult. If a certain tooth-cream penetrates, for example, well into the space between the diamond-shaped elements 32, the distances between the bristle structures 30 are too small to allow a sufficient and simple distribution of the toothpaste and with careless application the cleaning result can deteriorate.

The method provided by the present disclosure for cleaning and care of the teeth and/or the gums of a user, in particular for the prevention and/or treatment of diseases of the teeth, the dentines and/or the gums with a preferably also inventive-corresponding, mouth-, dental care and tooth cleaning-composition is summarized in FIG. 6.

The method according to the invention for cleaning and caring for the teeth and/or the gums should preferably be carried out at least twice a day, even more preferably two to four times a day, for example twice or three times a day.

As already explained in detail above, the present invention according to the first aspect relates to a dental care and tooth cleaning composition for use in a method for simultaneous cleaning of several, preferably all teeth of the upper and/or lower jaw, by means of a suitable tooth brushing device, wherein the dental care and tooth cleaning composition in foamed form on a mouth insert or several mouth inserts of the tooth brushing device is presented.

The applied oral-, dental care and tooth cleaning composition according to this first aspect is preferably the dental care and tooth cleaning composition of the invention according to the second aspect of the present invention. The preferred composition comprises:
15 to 30 wt.-%, preferably at least 20 wt.-%, even more preferably at least 22 wt.-%, glycerin,
optionally at least one other humectant, in addition to glycerin, wherein the at least one other humectant preferably sorbitol or hydrogenated starch hydrolysate, which preferably has a sorbitol content in the range of 65 to 75 wt.-%, is or comprises,
10 to 23.5 wt.-% at least one precipitated silicate,
0.2 to 4.0 wt.-% at least one surfactant,
0.05 to 0.5 wt.-% at least one viscosity amplifier, and
at least 10 wt.-% water.

Preferred embodiments of the method and use are described above in connection with the disclosure to the composition. Since these can be applied identically to the method and the use of the composition, reference is made to this.

The application of a low viscosity composition according to the present disclosure allows easy and precise application to a mouth insert. By the preferred application of the composition in the form of a foam, even more preferably in the form of a foam spray, in particular an improved adhesion to the cleaning surfaces in FIG. 1 is ensured, which are not shown, thus are directed downwards.

Likewise, the present invention relates to the use of the composition described herein for use in the field of oral and dental hygiene. Preferably, the composition is applied by means of a toothbrush device for the simultaneous cleaning of several, preferably all teeth of a user. The composition is presented according to this preferred embodiment by applying the composition to a mouth insert or to several mouth inserts of the toothbrush device.

### SHORT DESCRIPTION OF THE FIGURES

FIG. 1 shows an embodiment of a mouth insert of a tooth brushing device, which is suitable for the simultaneous cleaning of all teeth of a user and for the application of the composition according to the invention. The mouth insert comprises a coupling section for connecting the mouth insert with a drive device of the tooth brushing device (not shown).

FIG. 2 shows a supervision on a part of a mouth insert with cleaning structures contained therein.

FIG. 3 is a vertical longitudinal cross-sectional view of a tooth brushing device for the simultaneous cleaning of all teeth of a user within the meaning of the present invention.

FIG. 4A to 4D are each vertical longitudinal cross-sectional views of coupling-portions of different embodiments for a mouth insert of a tooth brushing device, which is suitable for simultaneous cleaning of all teeth of a user.

FIG. 5 is a perspective view of a support structure for another embodiment of a mouth insert of a tooth brushing device, which is suitable for simultaneous cleaning of all teeth of a user.

FIG. 6 is a flowchart of a method for the simultaneous cleaning of several, preferably all teeth of a user.

FIG. 7A to 7F show photographic images, in negative representation, for the foam stability of a composition according to the invention (each left) compared to a composition with low glycerin and water content (each right) over time. FIG. 7A shows the foams at time point 0 seconds, FIG. 7B shows the foams at the time point 30 seconds, FIG. 7C shows the foams at the time point 1 minute, FIG. 7D shows the foams at the time point 2 minutes, FIG. 7E shows the foams at the time point 3.5 minutes and FIG. 7F shows the foams at the time point 5 minutes.

FIG. 8 represents the decrease in the foam volume depending on the time. Comparing a composition according to the invention (marked as “invention”) and a composition with low glycerin and water content (marked as “reference”).

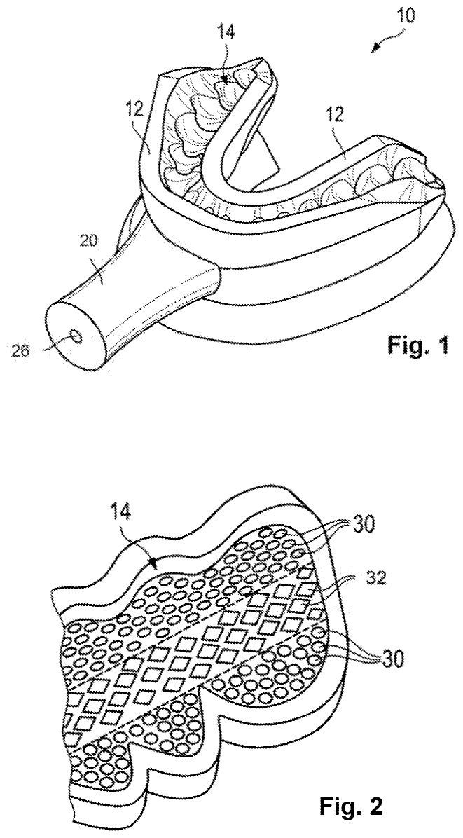

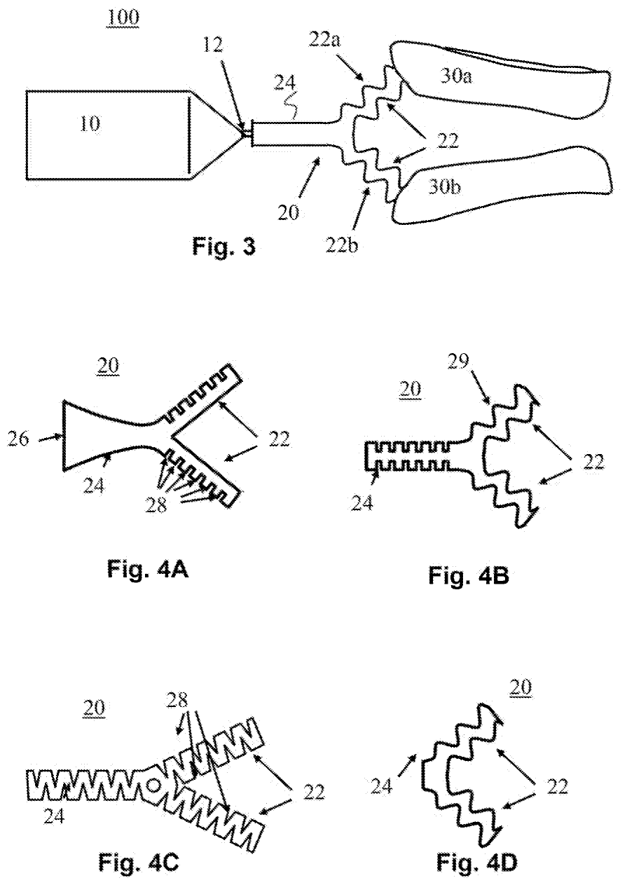

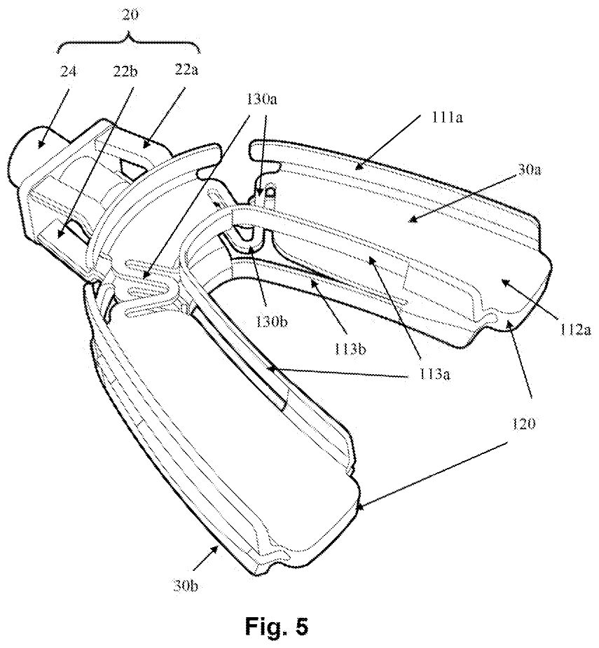

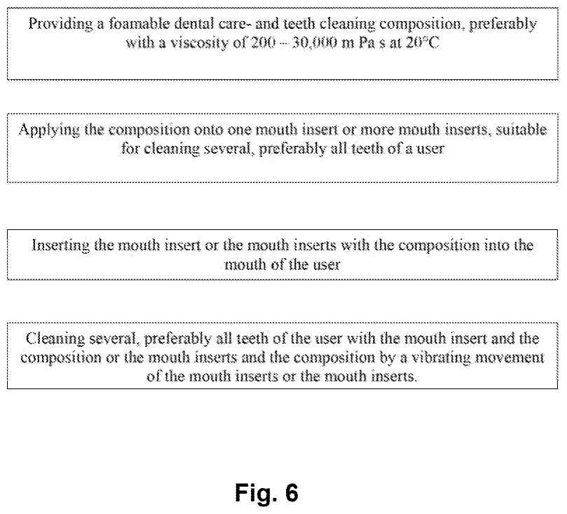

### MEASUREMENT METHODS

In the present invention, the determination of the viscosity is carried out with a Brookfield viscosimeter, preferably a rotary viscosimeter DV2T of the company Brookfield with heliopath T-spindles of spindle type C. The measurement is carried out at a speed of 10 rpm (revolutions per minute), 20° C. and normal pressure of 0.1 MPa (1013.25 mbar).

By measuring the torque required for the rotation of the spindle via the deflection of a spring, the viscosity of the composition according to the invention is determined.

The viscosity of the liquid composition is determined without propellant gas, i.e. at a time point before the composition according to a preferred embodiment is converted into a foam or an aerosol.

The viscosities or viscosity areas indicated above are therefore referred to in each case with a Brookfield viscometer, preferably a rotary viscosimeter DV2T of the company Brookfield, with heliopath T spindles of spindle type C at a speed of 10 rpm (revolutions per minute), 20° C. and normal pressure of 0.1 MPa (1013,25 mbar) measured dynamic viscosities in the composition without propellant gas.

The determination of the specific surface in (m²/g) according to the BET-method by the adsorption of gases, for example of nitrogen N₂, is carried out according to Brunauer, Emmett and Teller according to DIN 66131.

The average particle size of the silicates is determined using a Leeds and Northrup Microtrac II. A laser beam is projected through a transparent cell containing a stream of moving particles suspended in a liquid. Light rays that hit the particles are scattered around angles that are inversely proportional to their size. The photodetector arrangement measures the amount of light at several predetermined angles. Electrical signals, which are proportional to the measured luminous flux values, are then processed by a microcomputer system to form a multi-channel histogram of particle size distribution.

The RDA value is determined according to the method described in the article “A Laboratory Method for Assessment of Dentifrice Abrasivity”, John J. Hefferren, in the Journal of Dental Research, Vol. 55, No. 4 (1976), pages 563-573.

The brass Einlehner (BE) abrasion value or the Einlehner abrasion was measured by the use of a Einlehner AT-1000 abrasion tester. In this test, a Fourdrinier brass wire sieve is weighed and exposed to the effect of a 10%/aqueous silicate suspension for a certain number of revolutions and the abrasion quantity is then determined as a milligram material loss from the Fourdrinier wire sieve per 100,000 revolutions. The result is the 10% brass Einlehner-(BE) abrasion value.

The rotary body of the abrasion tester runs uniformly at 1800 rpm regardless of the power frequency (50/60 Hz). The silicate suspension is produced by mixing the silicate to be tested with deionized Wasser. For the preparation-a 10% suspension, for example, 100 g silicate are mixed with 900 g of water.

### EXAMPLES

### Example 1: Dental Care Composition

In a vacuum homogenizer, the following steps are performed at 6000 revolutions per minute to produce a basic dental care formulation:
1. Presentation of 1500 g hydrogenated starch hydrolysate with a sorbitol content of 70%
2. Addition of 100 g of olaflur (aminfluoride-containing raw material with an active substance content of 35%)
3. Addition of 2000 g or 1700 g glycerin
4. Homogenizing the mixture obtained in this way for 10 min
5. Addition of 500 g Zeodent 115
6. Addition of 300 g Zeodent 167
7. Addition of 500 g Zeodent 103
8. Homogenizing the mixture obtained in this way for 10 min
9. Addition of 6 g Xanthan Gum
10. Addition of 4 g acrylate cross polymer C10-C30
11. Homogenizing the mixture obtained in this way for 10 min
12. Addition of 50 g sodium lauryl sulfate
13. Addition of 10 g of cocamidopropyl betaine
14. Homogenizing the mixture obtained in this way for 10 min
15. Adding water ad 6000 g
16. Setting pH=7.0 with sodium hydroxide

During the filling process of the resulting composition in a bag of a bag-on-valve can, the composition is added 4.9 wt.-% cosmetic foaming machine (propellant gas), based on the propellant gas-containing composition.

The above-mentioned exemplary composition can be manually distributed in the mouthpieces by spraying as foam or aerosol therein. It has been shown that the composition is distributed very evenly in the recesses of the mouthpiece.

It has also been shown that in particular the cleaning substance, but also the other active substances are evenly distributed in the foam or aerosol. This ensures that local overdoses or underdosage are avoided. Thus, with the composition of the invention, very good cleaning results could be achieved.

The exemplary composition has also been advantageous in that it—and the foam formed from it adheres well and sufficiently long to the mouth piece to allow an insertion of the mouthpieces into the oral cavity, without it—or the foam running out of the mouth and back. This was particularly true for the composition or foam of the recess opened downwards for the absorption of the teeth of the lower jawline, in upside down position.

In the mouth, however, the foam disintegrates again.

The adhesion is measurable with a tensiometer.

### Example 2: Dental Care Composition

In a vacuum homogenizer, the following steps are performed at 6000 revolutions per minute to produce a basic dental care formulation:
1. Presentation of 1500 g sorbitol
2. Addition of 100 g of olaflur (aminfluoride-containing raw material with an active substance content of 35%)
3. Addition of 1700 g glycerin
4. Homogenizing the mixture obtained in this way for 10 min
5. Addition of 500 g Zeodent 115
6. Addition of 300 g Zeodent 167
7. Addition of 500 gZeodent 103
8. Homogenizing the mixture obtained in this way for 10 min
9. Addition of 6 g Xanthan Gum
10. Addition of 4 g acrylate cross polymer C10-C30
11. Homogenizing the mixture obtained in this way for 10 min
12. Addition of 50 g sodium lauryl sulfate
13. Addition of 10 g of cocamidopropyl betaine
14. Homogenizing the mixture obtained in this way for 10 min
15. Adding water ad 6000 g
16. Setting pH=7.0 with sodium hydroxide

During the filling process of the resulting composition in a bag of a bag-on-valve can, the composition is added 4.9 wt. % cosmetic foaming machine (propellant gas), based on the propellant gas-containing composition.

The above-mentioned exemplary composition can be manually distributed in the mouthpieces by spraying as foam or aerosol into the mouthpieces. It has been shown that the composition is distributed very evenly in the recesses of the mouthpiece.

It has also been shown that in particular the cleaning substance, but also the other active substances are evenly distributed in the foam or aerosol. This ensures that local overdoses or underdosage are avoided. Thus, with the composition of the invention, very good cleaning results could be achieved.

The exemplary composition has also been advantageous in that it- and the foam formed from it adheres well and sufficiently long to the mouth piece to allow an insertion of the mouthpieces into the oral cavity, without it- or the foam running out of the mouth and back. This was particularly true for the composition or foam of the recess opened downwards for the absorption of the teeth of the lower jawline, in upside down position. In the mouth, however, the foam disintegrates again.

The adhesion is measurable with a tensiometer.

### Example 3: Performance Test of Dental Care Composition

In order to check whether the compositions of the invention are suitable for application in a mouthpiece for simultaneous cleaning of all teeth of a user, an exemplary composition with a glycerin content of 25 wt.-% and a water content of 18 wt.-% was compared with a non-inventive composition containing 8 wt.-% glycerin and 9 wt.-% water.

Both compositions were applied from the bag-on-valve can in strand form to a glass plate and the foam adhesion and the decay of the foams were examined over time (0 seconds to 5 minutes).

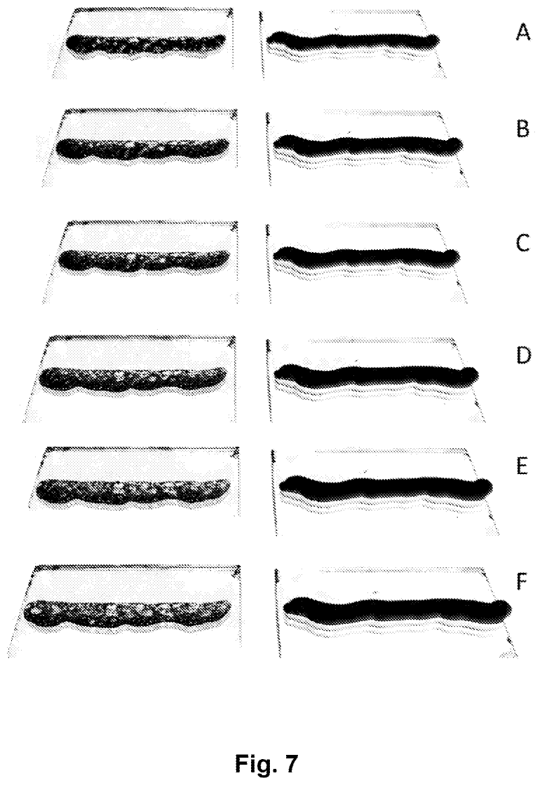

The results are shown in FIG. 7 A-F. The foam according to the invention (left) is characterized by a sufficiently long foam stability for applying the composition to a mouthpiece. However, the foam then disintegrates again as desired. In comparison, the non-inventive composition (right) foams very strongly and the foam does not collapse even after a long time.

### Example 4: Quantification of Foam Volume

The compositions according to Example 3 were filled into commercially available plastic measuring cylinders and the percentage decrease of the menisci (due to the foam decay) was determined depending on the time.

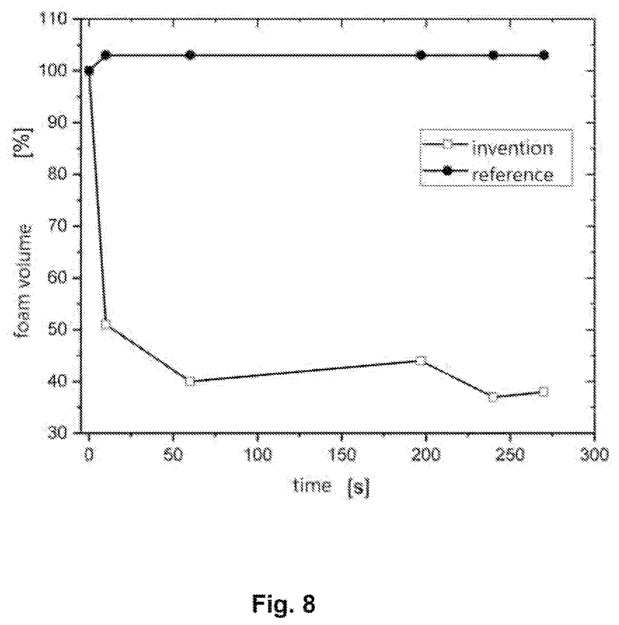

The results are shown in FIG. 8. It is confirmed that the composition according to the invention shows the desired short-term foam stability, but the foam then, as desired, disintegrates again.

What is claimed is:

1. A dental care and dental cleaning composition for use in a method of simultaneously cleaning a plurality of teeth of a upper and/or lower jaw by means of a dental brushing device suitable therefor, wherein the dental care and dental cleaning composition is presented in foamed form on at least one mouth insert of the dental brushing device, and wherein the dental care and teeth cleaning composition comprising:
10 to 40 wt % glycerol,
10 to 23.5 wt % of at least one precipitated silicate,
0.05 to 0.5 wt %, of at least one viscosity amplifier,
at least one surfactant in a proportion of 0.2 to 4.0 wt %, and
at least 10 wt % of water.

2. The dental care and dental cleaning composition for use according to claim 1 in a method for prevention and/or treatment of dental and gum diseases, in particular caries, periodontitis and/or plaque.

3. A dental care and dental cleaning composition comprising:
15 to 30 wt %, of glycerol,
at least one further humectant, besides glycerol, wherein the at least one further humectant is comprised sorbitol or hydrogenated starch hydrolysate having a sorbitol proportion in the range of 65 to 75 wt %,
10 to 23.5 wt % of a mixture of two or more precipitated silicates,
0.2 to 4.0 wt %, of at least one surfactant,
0.05 to 0.5 wt %, of at least one viscosity amplifier,
at least one active substance and/or at least one suitable excipient or additive, and
at least 10 wt % of water, wherein
the mixture of two or more precipitated silicates comprises first precipitated silicate with an average particle size in a of 7 to 8.5 μm and an Einlehner abrasion in the range of more than 5.0 to 25 mg loss/100 000 revolutions, second precipitated silicate with an average particle size in the range of more than 8.5 to 10 μm and an Einlehner abrasion in the range of more than 2.5 to 5.0 mg loss/100 000 revolutions, and third precipitated silicate with an average particle size in the range of more than to 14 μm and an Einlehner abrasion in the range of less than 2.5 loss/100 000 revolutions,
the composition does not include polyethyleneglycol or the proportion of polyethyleneglycol in the composition is at most 0.8 wt. %, and
the at least one viscosity amplifier is Xanthan Gum, and the proportion of cellulose or cellulose-derivates and/or algal polysaccharides, in particular Carragenaan, in the composition is at most 0 to 0.1 wt. %.

4. The dental care and dental cleaning composition according to claim 3, wherein the composition comprises as further humectant hydrogenated starch hydrolysate having a sorbitol proportion in the range of 65 to 75 wt %, or sorbitol, or the further humectant is hydrogenated starch hydrolysate having a sorbitol proportion in the range of 65 to 75 wt %, or sorbitol.

5. The dental care and dental cleaning composition according to claim 4, wherein the composition comprises 16.25% to 30 wt % of sorbitol.

6. The dental care and dental cleaning composition according to claim 3, wherein the composition comprises a viscosity in the range of 200 to 30,000 mPa s measured with a Brookfield DV2T rotational viscometer with Heliopath T spindles of spindle type C, at a rotational speed of 10 rpm, 20° C. and 1013.25 mbar.

7. (canceled)

8. (canceled)

9. The dental care and dental cleaning composition according to claim 3, wherein the composition does not include polyethylene glycol or the proportion of polyethylene glycol in the composition is at most 0.4 wt %.

10. The dental care and dental cleaning composition according to claim 3, wherein the proportion of cellulose or cellulose derivatives and/or algal polysaccharides, in particular Carragenaan, in the composition is in the range of 0 to 0.05 wt %.

11. The dental care and dental cleaning composition according to claim 3, wherein the composition further comprises at least one active substance selected from the group consisting of one or more compounds for remineralisation or ceramization such as hydroxyapatite, one or more fluoride active substances, a gum care agent, a disinfectant component and any mixture thereof, wherein the proportion of the one or more active substances in the composition is preferably 0.5 to 8 wt %.

12. The dental care and dental cleaning composition according to claim 3, wherein the composition further comprises:
16.25 to 30 wt % sorbitol,
15 to 30 wt %, glycerol,
1 to 2 wt % of amine fluoride-containing raw material with 20-50% active substance proportion,
6 to 13 wt. % precipitated silicate of category 1,
3 to 7 wt. % precipitated silicate of category 2,
1 to 3.5 wt. % precipitated silicate of category 3,
0.1-2 wt. % sodium lauryl sulphate,
0.1-2 wt. % cocamidopropyl betaine,
0.05-0.2 wt. % xanthan gum, and
at least 10 wt % of water.

13. The dental care and dental cleaning composition according to claim 3, wherein the composition comprises usual excipients and additives selected from the group consisting of flavourings, sweeteners, conserving agents, pigments, colourants, buffer substances and any mixture thereof, wherein the proportion of the one or more excipients and additives in the composition is preferably 0.1-5 wt %.

14. The dental care and dental cleaning composition according to claim 3, wherein the composition is in the form of a foam or an aerosol.

15. A system comprising a dental care and dental cleaning composition according to claim 3 and a dispenser having means for storing, conveying and dispensing the dental care and dental cleaning composition,
wherein the means for dispensing the composition comprises an outlet valve having an outlet opening, and
wherein the composition and the means for dispensing cooperate such that upon actuation of the outlet valve the dental cleaning composition exits the outlet opening in a stranded form.

16. The system of claim 15, wherein the means for conveying conveys the composition by means of a propellant gas or propellant gas mixture, and the means for dispensing is formed to be sufficiently pressure stable with respect to an expansion pressure of the propellant gas or propellant gas mixture.

17. The system according to claim 15, wherein the means for storing and dispensing the composition is an aerosol can or a bag-on-valve can.

18. The system according to claim 15, wherein the dental care and dental cleaning composition is provided in combination with a propellant gas or propellant gas mixture, in particular wherein the dental care and dental cleaning composition comprises a propellant gas or propellant gas mixture which is a compressible liquid having a boiling point of −10° C. or higher at normal pressure, or wherein the propellant gas or propellant gas mixture is not a compressible liquid, preferably dinitrogen oxide.

19. The system according to claim 18, wherein the propellant gas proportion of the composition is 0 to 15 wt %, or in the case of a non-compressible liquid, preferably dinitrogen oxide, the resulting pressure is in the range of 6 to 10 bar.

20. A method for simultaneously cleaning a plurality of teeth of a subject, wherein the tooth cleaning method comprising the following steps of:
providing a dental care and dental cleaning composition comprising 10 to 40 wt % glycerol, 10 to 23.5 wt % of a mixture of two or more precipitated silicates, 0.05 to 0.5 wt % of at least one viscosity amplifier, at least one surfactant having a proportion of 0.2 to 4.0 wt %, and at least 10 wt % water,
coating/applying the composition to at least one mouth insert of a vibratory tooth brushing device for the simultaneously cleaning of a plurality teeth of a user,
inserting the at least one mouth insert with the composition into a mouth of the user, and
cleaning the plurality of the user's teeth with the at least one mouth insert and the composition by activating the vibratory tooth brushing device.

21. The method according to claim 20, wherein the provided dental care and dental cleaning composition is a composition according to claim 3.

22. (canceled)

23. The dental care and dental cleaning composition according to claim 3 for use in a method of preventing and/or treating dental and gum diseases, in particular caries, periodontitis and plaque.
